SEQUENCE LISTING (44 sequences)

SEQ ID NO: 1 - DNA, 20 bp, Artificial Sequence
  primer for 27F
  misc_feature (12)..(12): m is a or c
agagtttgatcmtggctcag

SEQ ID NO: 2 - DNA, 22 bp, Artificial Sequence
  primer for 1492R
  misc_feature (6)..(6): y is c or t
tacggytaccttgttacgactt

SEQ ID NO: 3 - DNA, 19 bp, Artificial Sequence
  primer for 515F
  misc_feature (4)..(4): y is c or t
  misc_feature (9)..(9): m is a or c
gtgycagcmgccgcggtaa

SEQ ID NO: 4 - DNA, 20 bp, Artificial Sequence
  primer for 806R
  misc_feature (8)..(8): n is a, c, g, or t
  misc_feature (9)..(9): v is a, c or g
  misc_feature (14)..(14): w is a or t
ggactacnvgggtwtctaat

SEQ ID NO: 5 - DNA, 19 bp, Artificial Sequence
  primer for ITS1
tccgtaggtgaacctgcgg

SEQ ID NO: 6 - DNA, 20 bp, Artificial Sequence
  primer for ITS4
tcctccgcttattgatatgc

SEQ ID NO: 7 - DNA, 22 bp, Artificial Sequence
  primer for ITS1F
cttggtcatttagaggaagtaa

SEQ ID NO: 8 - DNA, 20 bp, Artificial Sequence
  primer for ITS2
gctgcgttcttcatcgatgc

SEQ ID NO: 9 - DNA, 20 bp, Artificial Sequence
  primer for GAPDH-F
ctctgctcctcctgttcgac

SEQ ID NO: 10 - DNA, 20 bp, Artificial Sequence
  primer for GAPDH-R
ttaaaagcagccctggtgac

SEQ ID NO: 11 - DNA, 20 bp, Artificial Sequence
  primer for Cldn3-F
cacgcgagaagaagtacacg

SEQ ID NO: 12 - DNA, 20 bp, Artificial Sequence
  primer for Cldn3-R
cctgcgtctgtcccttagac

SEQ ID NO: 13 - DNA, 20 bp, Artificial Sequence
  primer for Cldn4-F
catctcctctgttccgggta

SEQ ID NO: 14 - DNA, 20 bp, Artificial Sequence
  primer for Cldn4-R
atccactctgcacttcccag

SEQ ID NO: 15 - DNA, 20 bp, Artificial Sequence
  primer for Cldn7-F
gcaaaatgtacgactcggtg

SEQ ID NO: 16 - DNA, 19 bp, Artificial Sequence
  Cldn7-R
cacaaacatggccaggaag

SEQ ID NO: 17 - DNA, 20 bp, Artificial Sequence
  primer for Cldn12-F
gctgttttggaactgtcagg

SEQ ID NO: 18 - DNA, 20 bp, Artificial Sequence
  primer for Cldn12-R
ttccacacaggaaggaaagg

SEQ ID NO: 19 - DNA, 20 bp, Artificial Sequence
  primer for Ecad-F
gccgagagctacacgttcac

SEQ ID NO: 20 - DNA, 20 bp, Artificial Sequence
  primer for Ecad-R
gtcgagggaaaaataggctg

SEQ ID NO: 21 - DNA, 20 bp, Artificial Sequence
  primer for JamA-F
tcatattggcgatcctgttg

SEQ ID NO: 22 - DNA, 20 bp, Artificial Sequence
  primer for JamA-R
aggcacaggacaacttcaca

SEQ ID NO: 23 - DNA, 20 bp, Artificial Sequence
  primer for ZO1-F
acagcaatggaggaaacagc

SEQ ID NO: 24 - DNA, 20 bp, Artificial Sequence
  primer for ZO1-R
ccccactctgaaaatgagga

SEQ ID NO: 25 - DNA, 21 bp, Artificial Sequence
  primer for Muc1-F
ccctcccagtgtgcaaataag

SEQ ID NO: 26 - DNA, 20 bp, Artificial Sequence
  primer for Muc1-R
gaacggtgtcgtcgaaacag

SEQ ID NO: 27 - DNA, 19 bp, Artificial Sequence
  primer for TFF1-F
tgccgccgaaagaactacg

SEQ ID NO: 28 - DNA, 21 bp, Artificial Sequence
  primer for TFF1-R
tggggtactcgctcataggat

SEQ ID NO: 29 - DNA, 20 bp, Artificial Sequence
  primer for Tricel-F
ggcagctcggagacatagag

SEQ ID NO: 30 - DNA, 22 bp, Artificial Sequence
  Tricel-R
tttgctgttctcagttccttga

SEQ ID NO: 31 - DNA, 20 bp, Artificial Sequence
  primer for RPL37A-F
attgaaatcagccagcacgc

SEQ ID NO: 32 - DNA, 21 bp, Artificial Sequence
  primer for RPL37A-R
aggaaccacagtgccagatcc

SEQ ID NO: 33 - DNA, 22 bp, Artificial Sequence
  primer for Col1A1-F
gtcacccaccgaccaagaaacc

SEQ ID NO: 34 - DNA, 22 bp, Artificial Sequence
  primer for Col1A1-R
aagtccaggctgtccagggatg

SEQ ID NO: 35 - DNA, 20 bp, Artificial Sequence
  primer for COL6A3-F
aagccctgactggtatccct

SEQ ID NO: 36 - DNA, 20 bp, Artificial Sequence
  primer for COL6A3-R
cagccgcaccatttttgaca

SEQ ID NO: 37 - DNA, 21 bp, Artificial Sequence
  primer for HAS1-F
tcagcccaagattcttcagtc

SEQ ID NO: 38 - DNA, 20 bp, Artificial Sequence
  primer for HAS1-R
gaacgaggagaaagcaggac

SEQ ID NO: 39 - DNA, 20 bp, Artificial Sequence
  primer for DKK1-F
ccttggatgggtattccaga

SEQ ID NO: 40 - DNA, 20 bp, Artificial Sequence
  primer for DKK1-R
cctgaggcacagtctgatga

SEQ ID NO: 41 - DNA, 22 bp, Artificial Sequence
  primer for TNF?-F
atgagcactgaaagcatgatcc

SEQ ID NO: 42 - DNA, 22 bp, Artificial Sequence
  primer for TNF?-R
gagggctgattagagagaggtc

SEQ ID NO: 43 - DNA, 21 bp, Artificial Sequence
  primer for TL1A-F
cttccttgcaggactcaccac

SEQ ID NO: 44 - DNA, 22 bp, Artificial Sequence
  primer for TL1A-R
gctgatgtgaaggtgcaaactc

(19) United States
(12) Patent Application Publication
(10) Pub. No.: US 2020/0318162 A1
(43) Pub. Date: Oct. 8, 2020

(54) GUT BACTERIA IN PATIENTS WITH INFLAMMATORY BOWEL DISEASE AND METHODS THEREWITH IN DETECTING ILEAL FIBROSIS

(71) Applicant: Cedars-Sinai Medical Center, Los Angeles, CA (US)

(72) Inventors: Suzanne Devkota, Los Angeles, CA (US); Wing Yan Ha, Los Angeles, CA (US)

(73) Assignee: Cedars-Sinai Medical Center, Los Angeles, CA (US)

(21) Appl. No.: 16/832,977

(22) Filed: Mar. 27, 2020

Related U.S. Application Data
(60) Provisional application No. 62/825,689, filed on Mar. 28, 2019.

(51) Int. Cl. C12Q 1/689; G01N 33/68; C12Q 1/6883

(52) U.S. Cl. CPC C12Q 1/689; G01N 33/6893; G01N 2800/085; G01N 2800/065; C12Q 1/6883

(57) ABSTRACT

The present invention describes methods of identifying subjects susceptible to fibrosis by targeting bacteria found in intestine-related adipose tissue such as creeping fat, ileal mucosa or related tissue. Based on subject's susceptibility to developing fibrosis, different intervention or treatment options can be used.

### CROSS-REFERENCE TO RELATED APPLICATIONS

This application includes a claim of priority under 35 U.S.C. § 119(e) to U.S. provisional patent application No. 62/825,689, filed Mar. 28, 2019, the entirety of which is hereby incorporated by reference.

### REFERENCE TO SEQUENCE LISTING

The Sequence Listing submitted Mar. 27, 2020, as a text file named “Sequence Listing-065472-000745US00_ST25” created on Mar. 26, 2020 and having a size of 9,194 bytes, is hereby incorporated by reference.

### FIELD OF INVENTION

This invention relates to methods of detecting the development of fibrosis and treatment of fibrosis, particularly ileal fibrosis in Crohn's Disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Ileal fibrosis represents one of the major challenges in Crohn's disease (CD). Approximately 30-50% of patients suffering from CD will develop fibrotic strictures, and up to 75% of them will ultimately need surgery. Hospitalization and surgery are the largest contributor to the direct medical costs and indirect personal costs for this population, which further translates to an estimated $2.2 billion loss per year to the United States due to absence from the workforce. While therapies exist to manage inflammation in CD, none exist to prevent or manage fibrosis, leaving surgery as the only option. Despite surgical intervention, fibrotic strictures often recur. Thus, there remains a need in the art for methods of treating fibrosis and methods for early detection and detection of the development of fibrosis, particularly in CD patients or individuals who are at a higher risk of having CD.

Despite meaningful advances in IBD treatment and care, ileal CD patients suffering from recurrent fibrotic disease are among the most numerous population with the fewest treatment options. This represents an enormous unmet need. The invention described herein will not only impact these individuals, but also fibrosis-mediated diseases broadly.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A shows a depiction of normal mesenteric adipose tissue (MAT) versus creeping fat (CrF).

FIGS. 1B and 1C show two directly adjacent samples from the same patient. FIG. 1B: Healthy ileum and attached mesenteric adipose tissue from a resection sample in our preliminary cohort. FIG. 1C: Inflamed and fibrotic ileum surrounded by creeping fat.

FIG. 1D shows a cartoon of sampling sites in a Crohn's disease patient. The same site pairings were sampled for ulcerative colitis resections as a control.

FIG. 1E is a schematic of experimental design and read outs from host and microbial profiling. Cultivation combined with sequencing were used to determine microbial candidates for creeping fat development. Single-cell RNA sequencing was used to better understand the cellular phenotype of creeping fat. Serum analysis and co-culture assays involving patient derived primary cells were included for validation of putative host-microbe links.

FIG. 2A depicts relative abundance of bacterial phyla detected by 16S rRNA sequencing in involved and uninvolved mucosa (I-MUC and U-MUC, respectively), involved and uninvolved mesenteric adipose tissue (I-MAT and U-MAT, respectively) from Crohn's disease (CD) and ulcerative colitis (UC) patients.

FIG. 2B shows absolute abundance of these phyla in each tissue.

FIG. 3A depicts key cultivable organisms recovered from mesenteric adipose tissue. Organisms found in one or more samples and their closely related species are shown. Each column represents the cultivable community for an individual patient. Detected organisms are colored and color intensity reflects the organism's overall association with Crohn's disease (CD) (black) vs. ulcerative colitis (UC) (light grey) specimen, or both (mid grey).

FIG. 3B depicts isolates from creeping fat are distinct from those recovered from uninvolved mesenteric adipose tissue as shown by pure culture assays (Bottom). Biolog assay to assess growth on different substrates was conducted in minimal medium containing various substrates as the sole nutrient source. OD600 values shown for each strain are the averaged obtained from two independent experiments.

FIG. 4 shows motility of Clostridium innocuum isolates. On the left and in the middle are motility test of C. innocuum in pre-reduced brain-heart infusion media with 0.3% agar. Motility is designated by growth deviated from the center stab line after 48 hours. On the right is non-motile bacteria, Staphylococcus aureus, is included here as negative control.

FIG. 5A shows single-cell RNA sequencing t-SNE plot that reveals distinct cellular clustering between Crohn's disease (CD) and ulcerative colitis (UC). FIG. 5B shows t-SNE plot of total cells in combined mesenteric adipose tissue and creeping fat from CD only. Numbers represent cell populations from most abundant ‘0’ to least abundant ‘9’. FIG. 5C shows t-SNE of cell populations comparing creeping fat to uninvolved mesenteric adipose tissue.

FIGS. 5D and 5E show the top 20 genes expressed in each cell cluster in the panel in FIG. 5C. FIG. 5D shows clusters 0-3, and FIG. 5E shows clusters 4-7. This highlights the high expression of extracellular matrix gene expression across almost all clusters (pro-fibrosis genes), and the high expression of microbe-sensing genes (showing these bacteria can persist in adipose and affect host cellular phenotypes).

FIGS. 6A and 6B depict qRT-PCR analysis of tight junction genes from the ileum of Crohn's diseases (CD) and colon of ulcerative colitis (UC). FIG. 6A depicts the mean of the inflamed sample data normalized to each sample's paired uninvolved specimen. In other words, CD and UC are both presented on the graph, but normalized to each disease's healthy control (dotted line). FIG. 6B depicts the same data, but normalized to the CD healthy control. This data allows for comparison of CD vs UC. Data below the dotted line represents reduced expression of target genes and increased permeability. Gene expression was measured by RT-qPCR. (n=10 for CD; n=8 for UC). Data are presented as mean±SEM. Two-way ANOVA with post hoc Bonferroni test or Mann-Whitney U test; **p<0.01.

FIG. 6C shows results from ELISA for plasma lipopolysaccharide-binding protein (LBP) level, in Crohn's disease (CD, n=14) and ulcerative colitis (UC, n=11). LBP is a blood marker for intestinal permeability, and in this case shows less systemic dissemination of bacteria due to the presence of creeping fat as a biocontainment barrier which is absent in UC.

FIGS. 7A and 7B depicts markers of M1- and M2-like macrophages determined by ELISA (IL-1β and TGF-β) and RT-qPCR (TNF-α). Results were derived from pooled macrophages from three CD patients. The results indicate Clostridium innocuum drives M2 macrophage polarization and exacerbated wound-healing response in proliferative cells. FIG. 7B shows the same experiment measured using macrophages from healthy controls. Similar trends between CD and healthy controls indicate consistent pro-fibrotic M2 effect of Clostridium innocuum similar to the IL-4 positive control for M2 differentiation.

FIG. 7C depicts co-culture of stem cells and fibroblasts from CD patients with bacterial lysates in the presence or absence of culture media supernatant (S/N) containing secreted products from stimulated macrophages (characterized in FIG. 7A-7B). Changes in expression of genes related to extra-cellular matrix production and Wnt signaling pathway were determined by RT-qPCR.

FIGS. 8A-8F depict bacterial and fungal sequencing reveal microbial DNA in the mesenteric adipose tissue are of gut origin. FIG. 8A shows community richness of the MAT- and mucosa (MUC)-associated microbiota in different subtypes of IBD. CD=Crohn's disease, UC=ulcerative colitis. FIG. 8B shows community richness of the MAT- and MUC-associated mycobiota in different subtypes of IBD. FIGS. 8C-8F show relative abundances of dominant fungal signals (Saccharomyces cerevisiae in 8C, Malassezia restricta in 8D, Malassezia globosa in 8E, and Candida metapsilosis in 8F) in the adipose and mucosal regions of CD and UC resections.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

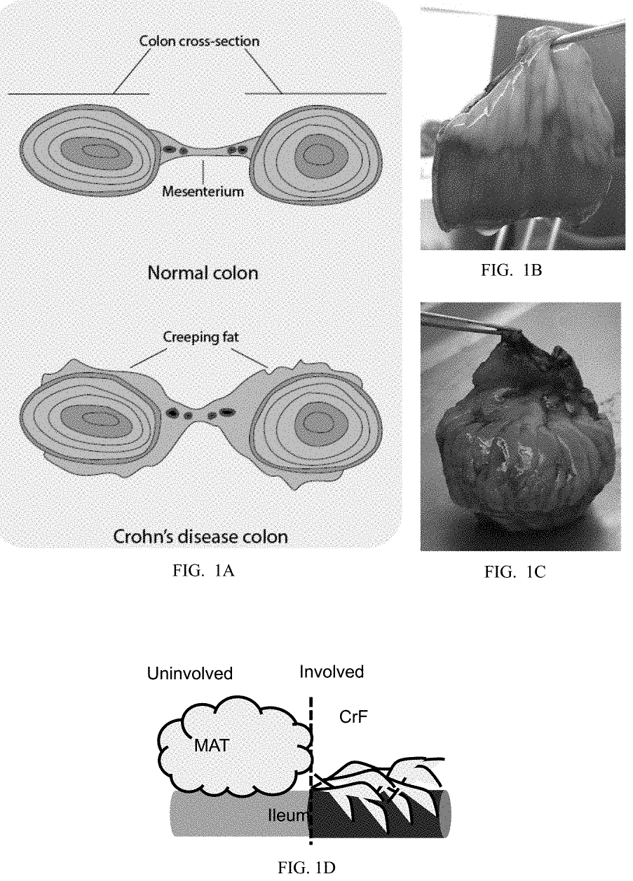

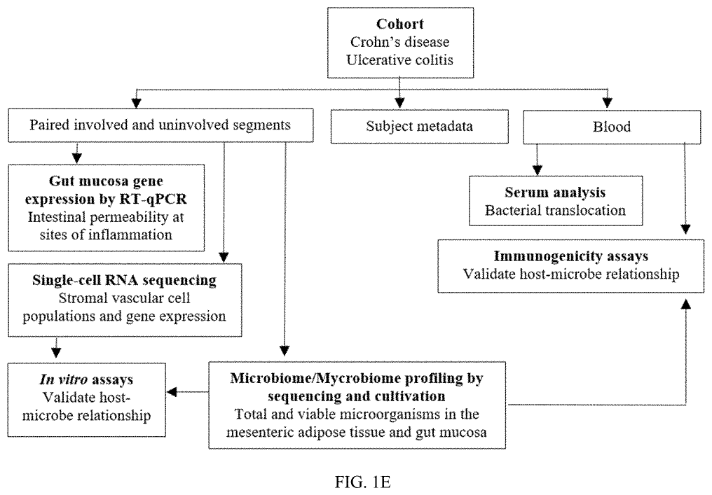

Creeping fat (CrF) is an extra-intestinal manifestation of Crohn's Disease often found in patients with stricturing complications, and presents as hyperplastic mesenteric adipose tissue (MAT) which expands and wraps specifically around sites of intestinal inflammation, primarily in the small bowel, and most often the ileum. CrF is not found in ulcerative colitis (UC), the other form of inflammatory bowel diseases (IBD). CrF is also observed in patches, extending like fingerlike projections gripping the inflamed (involved) segments of intestine, adjacent to normal (uninvolved) MAT, which is pliable and attached longitudinally to a single border (FIG. 1A). CrF itself and the underlying intestinal tissue tend to be severely fibrotic, with the adipose encroachment into the intestinal muscularis (FIG. 1B, 1C). Despite surgical intervention, fibrotic strictures often recur.

Abbreviations: AAT abdominal wall adipose tissue, CD Crohn's Disease, CrF creeping fat, EBV Epstein-Barr virus, ECM extracellular matrix, ASCA anti-Saccharomyces cerevisiae antibody, CD Crohn's Disease, CBir bacterial flagellin, ddseq digital drop seq, HSP heat shock protein, IBD inflammatory bowel diseases, IBDGC inflammatory bowel disease genetics consortium, IEC intraepithelial lymphocyte, ITS internal transcribed spacer, LP lamina propria, MAT mesenteric adipose tissue, MSC mesenchymal stem cells, OmpC outer membrane protein C, PRS polygenic risk score, ScRNA-seq single-cell RNA sequencing, UC ulcerative colitis, ASCs adipose-derived mesenchymal stromal cells, EMC extracellular matrix.

The recurrence of fibrosis leads us to believe that there are persistent features of the ileal milieu leading to fibrosis that are not fully understood. One consistent yet poorly described feature of ileal CD is the presence of “creeping fat” (CrF) surrounding the sites of inflammation, and absence from healthy adjacent tissue. This visually remarkable characteristic is a form of hyperplastic mesenteric adipose tissue (MAT), which to our knowledge, has not been studied in the context of CD fibrosis.

We aimed to understand whether CrF was a pro-inflammatory or protective mechanism in CD. While not wishing to be bound by any particular theory, we believe that either outcome was a response to microbial signals resulting from bacterial and/or fungal translocation from the intestines to the MAT. We also believed that these microbiota would differ from those found in neighboring healthy MAT, or from MAT in another form of inflammatory bowel diseases (IBD), ulcerative colitis (UC). Studies have attempted to characterize CrF using in vitro cultivation, histology, or imaging, but have neither explored the role of the microbiome nor attempted to define the exact cellular make-up of the tissue. Findings from our study described herein included the discovery that many bacteria can translocate from the intestines to the MAT, however one organism, Clostridium innocuum (also known as [Eubacterium] dolichum), was most abundant and consistently translocated in mesenteric adipose samples. This led us to characterize the cellular composition and transcriptional activity of CrF versus uninvolved MAT in CD, and versus MAT in UC, using single-cell RNA sequencing. If differential gene expression was observed between CrF and these other states of MAT it can indicate an influence of C. innocuum. During the course of this study we observed CrF itself was fibrotic to the touch and, at a cellular level, discovered it is transcriptionally upregulated for extracellular matrix production in nearly every cell type, but primarily mesenchymal stem cells (MSCs). However, there was not a related increase in gene expression for classic pro-inflammatory mediators. In fact, there appeared to be a downregulation of stress responses, primarily of heat shock proteins (HSPs). This transcriptional profile was not shared by the other forms of MAT. This indicates that CrF may initially be a protective response to chronic ileal inflammation, but some other factor drives a constitutively active wound healing response manifested as extracellular matrix deposition and fibrosis. This factor may be driven by C. innocuum, host genetics, or a combination of the two.

This discovery is significant because we have, for the first time, identified both a microbial target and cellular pathway in CrF that we believe influence and/or mimic the fibrotic cascade present in the ileum of CD patients. We identify a cultivable, gut-derived bacterial and fungal community in human mesenteric adipose tissue, with a microbial signature that distinguishes CD from UC adipose, despite co-occurrence of these organisms in the mucosa of both the ileum and colon. We have further identified a specific bacterium that distinguishes CrF from uninvolved MAT.

IBD has classically been divided into either UC or CD, and genome-wide association studies (GWAS) have failed to distinguish these two diseases, much less sub-phenotypes within each disease. Recently, the largest IBD genotype-phenotype study to-date in 29,838 IBD patients from 49 centers in Europe, North America, and Australasia concluded that this IBD classification is too broad, and instead should consider ileal and colonic CD as two separate disease entities. This conclusion was based on polygenic risk scores incorporating genotype-phenotype associations across 156,154 genetic variants. Predictive models utilizing these genetic risk scores strongly distinguished ileal from colonic CD and support the use of genetic risk scores to characterize sub-phenotypes. Furthermore, this study concluded that disease location is an intrinsic part of a patient's disease and is in-part genetically determined. This supports the need for deeper characterization of sub-phenotypes and integrated phenotype-genotype models that take into account genetics along with additional host factors such as the microbiome and gene expression.

Our data as described herein has generated the first single cell whole transcriptome data set on both CrF and MAT and has revealed new cellular activities and interactions never before described in this tissue. We have, for the first time, identified a CrF microbiome dominated by a single organism, C. innocuum. We have, for the first time, recovered cultivable fungi from CrF and MAT across CD and UC, indicating that interaction between bacteria and fungi in the peri-intestinal environment is relevant to disease.

Various embodiments the present invention are based, at least in part, on these findings.

### Examination and Assaying

Various embodiments provide for a method of conducting an examination of a subject, comprising: detecting for the presence or absence of a microorganism in a biological sample of the subject, and the microorganism comprises, or consists of, one or more of Clostridium, Parabacteroides, Erysipelatoclostridium, Veillonella, and Bifidobacteria.

Various embodiments provide for a method of conducting an assay for a subject, wherein the assay comprises detecting for the presence or absence of a microorganism in a biological sample of the subject, and the microorganism comprises, or consists of, one or more of Clostridium, Parabacteroides, Erysipelatoclostridium, Veillonella, and Bifidobacteria.

In some embodiments, the microorganism for assay or detection is any one, two, three, four, five or all six of Clostridium innocuum, Parabacteroides distasonis, Erysipelatoclostridium ramosum, Clostridium symbiosum, Veillonella parvula, and Bifidobacteria pseudolongum.

In some embodiments, the microorganism for assay or detection is Clostridium innocuum.

In some embodiments, the microorganism further comprises any one or more of Bacteroides vulgatus, Ruminococcus gnavus, Tyzzerella nexilis, and Klebsiella. In some embodiments, the microorganism further comprises any one or more of Escherichia/Shigella, Arabia massiliensis, Bifidobacteria bifidum, Collinsella aerofaciens, Bacteroides cellulosilyticus, Bacteroides fragilis, Bacteroides thetaiotaomicron, Dorea longicatena, Ruminococcus torques, Eggerthella lenta, Lactobacillus gasseri, Flavonifractor plautii, Bilophila wadsworthia, Bifidobacteria longum, and Blautia obeum.

Yet in some embodiments, the microorganism does not comprise any one or more of Bacteroides vulgatus, Ruminococcus gnavus, Tyzzerella nexilis, Klebsiella, Escherichia/Shigella, Arabia massiliensis, Bifidobacteria bifidum, Collinsella aerofaciens, Bacteroides cellulosilyticus, Bacteroides fragilis, Bacteroides thetaiotaomicron, Dorea longicatena, Ruminococcus torques, Eggerthella lenta, Lactobacillus gasseri, Flavonifractor plautii, Bilophila wadsworthia, Bifidobacteria longum, and Blautia obeum. In some embodiments, the microorganisam does not comprise any one or more of those otherwise listed in Table 3. In some embodiments, the method does not include detecting the presence or absence of any one or more of Bacteroides vulgatus, Ruminococcus gnavus, Tyzzerella nexilis, Klebsiella, Escherichia/Shigella, Arabia massiliensis, Bifidobacteria bifidum, Collinsella aerofaciens, Bacteroides cellulosilyticus, Bacteroides fragilis, Bacteroides thetaiotaomicron, Dorea longicatena, Ruminococcus torques, Eggerthella lenta, Lactobacillus gasseri, Flavonifractor plautii, Bilophila wadsworthia, Bifidobacteria longum, and Blautia obeum. In some embodiments, the method does not include detecting the presence or absence of any one or more of the microorganisms otherwise listed in Table 3.

In some embodiments, detecting the presence of the microorganism includes detecting a measurable amount of the microorganism that is greater than the sensitivity limit of the assay or technique used for the detection. In some embodiments, detecting the presence of the microorganism includes detecting an abundance of the presence of the bacterial microorganism, e.g., the microorganism is in a quantity of at least 5%, 6%, 7%, 8%, 9%, 10%, 20%, or 30%, e.g., in number, compared to the total quantities of all bacterial microorganisms in the biological sample.

In other embodiments, detecting the presence of the microorganism further includes obtaining the biological sample from the subject. In one embodiment, obtaining the biological sample includes collecting the biological sample from the subject. In another embodiment, obtaining the biological sample includes receiving a collected biological sample.

### Subject Populations

In various embodiments, the subject is a human. In other embodiments, the subject is a mammal, such as dog, cat, horse, cow, bull, or pig. In some embodiments, the subject is a human having or suspected of having an inflammatory bowel disease. In some embodiments, the subject is a human having or suspected of having Crohn's disease. In some embodiments, the subject is one who desires to know whether he or she has fibrosis. In some embodiments, the subject is one who desires to know whether he or she has ileal fibrosis or is at risk for developing ileal fibrosis. In some embodiments, the fibrosis is pulmonary fibrosis, liver fibrosis, heart fibrosis, mediastinal fibrosis, retroperitoneal fibrosis, skin fibrosis, adipose fibrosis, or scleroderma.

In some embodiments, the subject does not have ulcerative colitis. In some embodiments, the subject is a human over 25 years old, over 30 years old, over 40 years old, over 50 years old, over 60 years old, over 70 years old, or over 80 years old. In some embodiments, the subject is between 25 and 30 years old, between 30 and 35 years old, between 35 and 40 years old, between 45 and 45 years old, between 45 and 50 years old, between 50 and 60 years old, between 60 and 70 years old, between 70 and 80 years old, between 85 and 90 years old, or in an age range between any two integers mentioned above. In some embodiments, the subject is in adulthood or a senior.

In some embodiments, the subject has symptoms of or is diagnosed with Crohn's disease, but has not shown signs of ileal fibrosis, e.g., before the onset of fibrotic strictures. In some embodiments, the subject has recently been diagnosed with Crohn's disease in the past 6 months, 1 year, 2 years, or 3 years; and/or the subject desires to be tested for the likelihood and/or the severity of fibrosis.

In other embodiments, the subject has or has had Crohn's disease, and is undergoing treatment and/or has undergone treatment for removal of ileal fibrosis, including surgical procedures, and the subject is examined or assayed for the presence or absence of the microorganism so as to determine or assess the efficacy of the treatment. In one embodiment, the subject detected with the presence of the microorganism is directed to further treatment for fibrosis mitigation or removal. In another embodiment, the subject detected as not having the microorganism does not continue with the treatment.

### Biological Samples

In various embodiments, the biological sample is stool, mucosal biopsy (e.g., mucosa), mucosal wash, or adipose tissue of the subject. In various embodiments, the biological sample is stool, mucosal biopsy or mucosal wash. In various embodiments, mucosal wash is the mucosal debris that is collected when water is flushed against the bowel wall. The mucosal wash can a clinical sample that is like a hybrid of a mucosal biopsy and a stool sample. In various embodiments, the adipose tissue is abdominal wall adipose tissue, creeping fat, or mesenteric adipose tissue.

Additional examples of biological samples include but are not limited to body fluids, whole blood, plasma, stool, intestinal fluids or aspirate, intestinal mucosal biopsies, fat biopsies, and stomach fluids or aspirate, serum, cerebral spinal fluid (C SF), urine, sweat, saliva, pulmonary secretions, breast aspirate, prostate fluid, seminal fluid, cervical scraping, amniotic fluid, mucous, and moisture in breath. In particular embodiments of the method, the biological sample may be whole blood, blood plasma, blood serum, stool, intestinal fluid or aspirate or stomach fluid or aspirate.

### Detection Methods

In various embodiments, detection of the microorganisms can be performed by gene sequencing, cultivation on various liquid and solid media, mass spectrometry, polymerase chain reaction, and/or colorimetric/biochemical assays.

### Detection and Diagnostics

Various embodiments provide for a method of detecting fibrosis or determining the likelihood of developing fibrosis, comprising: obtaining a biological sample from a subject in need thereof; and detecting for the presence of absence of a microorganism in the biological sample of the subject in need thereof, wherein the microorganism comprises, or is selected from the group consisting of, Clostridium, Parabacteroides, Erysipelatoclostridium, Veillonella, and Bifidobacteria, and combinations thereof. Further embodiments provide that the biological sample can be from stool, mucosal biopsy, mucosal washing, or mesenteric adipose tissue. The presence of these microorganisms in biological samples such as stool, mucosal biopsy or mucosal washing indicates there is a high likelihood that these microorganisms will translocate to, or are present in, the mesenteric adipose tissue and cause creeping fat.

In some embodiments, the microorganism for the detection or determination (diagnosis) is any one, two, three, four, five or all six of Clostridium innocuum, Parabacteroides distasonis, Erysipelatoclostridium ramosum, Clostridium symbiosum, Veillonella parvula, and Bifidobacteria pseudolongum. In some embodiments, the microorganism for the detection or determination (diagnosis) is Clostridium innocuum. In further embodiments, the microorganiam for the detection or determination (diagnosis) further comprises any one or more of those otherwise listed in Table 3. Yet in some embodiments, the microorganiam for the detection or determination (diagnosis) comprises any one, two, three, four, five or all six of Clostridium innocuum, Parabacteroides distasonis, Erysipelatoclostridium ramosum, Clostridium symbiosum, Veillonella parvula, and Bifidobacteria pseudolongum, but does not comprise any one or more of those otherwise listed in Table 3.

Various embodiments provide for a method of detecting the presence or absence of a microorganism selected from the group consisting of Clostridium, Parabacteroides, Erysipelatoclostridium, Veillonella, and Bifidobacteria, and combinations thereof in a biological sample of a subject, comprising: obtaining a biological sample from a subject in need thereof; and detecting for the presence or absence of a microorganism selected from the group consisting of Clostridium, Parabacteroides, Erysipelatoclostridium, Veillonella, and Bifidobacteria in the biological sample of the subject desiring to determine whether fibrosis is present, or whether fibrosis will develop.

In some embodiments, the method of detecting the presence or absence of the microorganism further comprises selecting a subject having an inflammatory bowel disease, and obtaining from the subject the biological sample for the detection. In some embodiments, the method of detecting the presence or absence of the microorganism further comprises selecting a subject having Crohn's disease, and obtaining from the subject the biological sample for the detection. In some embodiments, the method of detecting the presence or absence of the microorganism is for a subject not having ulcerative colitis.

Various embodiments provide for a method of detecting the presence or absence of a microorganism selected from the group consisting of Clostridium innocuum, Parabacteroides distasonis, Erysipelatoclostridium ramosum, Clostridium symbiosum, Veillonella parvula, and Bifidobacteria pseudolongum, and combinations thereof in a biological sample of a subject, comprising: obtaining a biological sample from a subject in need thereof; and detecting for the presence or absence of a microorganism selected from the group consisting of Clostridium innocuum, Parabacteroides distasonis, Erysipelatoclostridium ramosum, Clostridium symbiosum, Veillonella parvula, and Bifidobacteria pseudolongum in the biological sample of the subject desiring to determine whether fibrosis is present, or whether fibrosis will develop.

In one embodiment, a method of detecting the presence or absence of a microorganism selected from the group consisting of Clostridium innocuum, Parabacteroides distasonis, Erysipelatoclostridium ramosum, Clostridium symbiosum, Veillonella parvula, and Bifidobacteria pseudolongum and combinations thereof in adipose tissue of a subject, comprises: obtaining an adipose tissue sample from a subject in need thereof; and detecting for the presence or absence of a microorganism in the adipose tissue sample of the subject desiring to determine whether fibrosis is present, or whether fibrosis will develop.

In another embodiment, a method of detecting the presence or absence of a microorganism selected from the group consisting of Clostridium innocuum, Parabacteroides distasonis, Erysipelatoclostridium ramosum, Clostridium symbiosum, Veillonella parvula, and Bifidobacteria pseudolongum, and combinations thereof in a biological sample of a subject, said biological sample comprising stool, mucus or mucosal secretion, wherein the method comprises obtaining a biological sample from a subject in need thereof; and detecting for the presence or absence of a microorganism in the biological sample of the subject desiring to determine whether fibrosis is present or whether fibrosis (e.g., ileal fibrosis) will develop. The presence of these microorganisms in one of these biological samples indicates there is a high likelihood that these microorganisms are present in the mesenteric adipose tissue or creeping fat.

In some embodiments, the presence of these microorganisms in one of these biological samples for a subject having the inflammatory bowel disease in the methods indicates the subject is having Crohn's disease, rather than ulcerative colitis.

In some embodiments, the absence of these microorganisms in one of these biological samples for a subject having the inflammatory bowel disease in the methods indicates the subject does not have Crohn's disease.

In various embodiments, the microorganism that is detected is selected from the group consisting of Clostridium innocuum, Parabacteroides distasonis, Erysipelatoclostridium ramosum, Clostridium symbiosum, Veillonella parvula, Bifidobacteria pseudolongum, and combinations thereof. In particular embodiments, the microorganism that is detected is Clostridium innocuum.

In various embodiments, the fibrosis is ileal fibrosis.

In various embodiments, the fibrosis is pulmonary fibrosis, liver fibrosis, heart fibrosis, mediastinal fibrosis, retroperitoneal fibrosis, skin fibrosis, adipose fibrosis, or scleroderma.

In various embodiments, the subject has Crohn's disease. In various embodiments, the subject is suspected of having Crohn's disease.

Other embodiments provide the methods further comprise detecting in the biological sample the level of expression of an oxidative stress-related gene or protein, an adhesion-related gene or protein, an iron acquisition-related gene or protein, a lipid metabolism-related gene or protein, and/or a metabolite, wherein the oxidative stress-related gene or protein comprises superoxide dismutase, superoxide reductase, peroxiredoxin/thiol peroxidase, NADH oxidase, cystathionine beta-lyases/cystathionine gamma-synthases, manganese catalase, or Alkyl hydroperoxide reductase/anaerobic sulfite reductase; the adhesion-related gene or protein comprises ethanolamine permease, tryptophan synthase, peptidase M23/37, fibronectin, fibrinogen-binding protein, oligopeptide ABC transporter substrate-binding protein OppA, oligopeptide ABC transporter ATP-binding protein OppD, oligopeptide ABC transporter ATP-binding protein OppF, or type IV fimbriae; the iron acquisition-related gene or protein comprises ABC-type Fe3+ transport system or iron ABC transporter permease; the lipid metabolism-related gene or protein comprises monoglyceride lipase or lysophospholipase; and the metabolite comprises acyl-CoA:acetate CoA-transferase, acetate kinase, or tryptophan synthase.

Other embodiments provide for a method of determining the likelihood of developing fibrosis in a subject having an inflammatory bowel disease, comprising obtaining a biological sample selected from stool, mucosa, or mucosal wash of the subject, isolating bacterial microorganisms from the biological sample, and detecting for the motility of one or more isolated microorganisms, wherein the subject is determined to have a high likelihood of developing the fibrosis if the isolated microorganisms comprises one or more of Clostridium innocuum, Parabacteroides distasonis, Erysipelatoclostridium ramosum, Clostridium symbiosum, Veillonella parvula, and Bifidobacteria pseudolongum, and/or motility is detected in one or more of these isolated microorganisms.

### Methods of Treatment

Various embodiments of the present invention provide for a method of treating fibrosis, comprising: administering an agent to eradicate a microorganism in adipose tissue to a subject in need thereof, wherein the microorganism is selected from the group consisting of Clostridium, Parabacteroides, Erysipelatoclostridium, Veillonella, and Bifidobacteria, and combinations thereof, whereby the microorganism is eradicated or partially eradicated.

Various embodiments of the present invention provide for a method of treating fibrosis, comprising: administering an agent to eradicate a microorganism in adipose tissue to a subject in need thereof, wherein the microorganism is selected from the group consisting of Clostridium, Parabacteroides, Erysipelatoclostridium, Veillonella, and Bifidobacteria, and combinations thereof, whereby the microorganism is eradicated or partially eradicated, wherein the subject in need thereof has been determined to have the microorganism in a biological sample obtained from the subject. The biological sample can be those as described herein.

Various embodiments of the present invention provide for a method of diagnosing a subject with Crohn's disease and treating the subject, comprising: detecting in a biological sample of the subject for the presence of a microorganism selected from the group consisting of Clostridium, Parabacteroides, Erysipelatoclostridium, Veillonella, and Bifidobacteria, and combinations thereof, and administering an agent to the subject so as to eradicate the microorganism.

Various embodiments provide for a method of determining the likelihood of developing fibrosis in a subject having an inflammatory bowel disease and treating the subject, comprising: detecting in a biological sample of the subject for the presence or absence of a microorganism selected from the group consisting of Clostridium innocuum, Parabacteroides distasonis, Erysipelatoclostridium ramosum, Clostridium symbiosum, Veillonella parvula, Bifidobacteria pseudolongum, and combinations thereof, wherein the presence of one or more of these microorganism indicates the subject is likely to develop fibrosis, and the absence of any of these microorganism indicates the subject is not likely to develop fibrosis; and treating the subject indicated to be likely to develop fibrosis with an inhibitor of type IV pili or an agent that prevents or mitigates fibrosis, optionally in addition to administering to the subject a medication for the inflammatory bowel disease; wherein the method does not administer an inhibitor of type iv pili to a subject not indicated likely to develop fibrosis.

In various embodiments, the microorganism that is eradicated or partially eradicated is selected from the group consisting of Clostridium innocuum (also known as [Eubacterium] dolichum), Bacteroides vulgatus, Parabacteroides distasonis, Erysipelatoclostridium ramosum (formerly known as Clostridium ramosum), Clostridium symbiosum, Veillonella parvula, Ruminococcus gnavus, and combinations thereof. In particular embodiments, the microorganism is Clostridium.

In various embodiments, the adipose tissue is abdominal wall adipose tissue, creeping fat, or mesenteric adipose tissue.

In various embodiments, the agent is a prebiotic, probiotic, or a mixture of both, and the agent eradicates or partially eradicates the microorganism by inhibiting the growth of the microorganism.

Examples of probiotics include but are not limited to Bifidobacteria, Lactobacillius, Saccharomyces boulardii. Examples of prebiotics include but are not limited to Inulin and Fructooligosaccharides (FOS).

In various embodiments, the agent is a drug capable of specifically inhibiting the growth of the microorganism or is specifically cytotoxic to the microorganism.

In various embodiments, an inhibitor of type iv pili is trifluoperazine or a phenothiazines (e.g. thioridazine).

In various embodiments, the agent is an antibiotic. Examples of antibiotics include, but are not limited to rifaximin, neomycin, metronidazole, which substantially non-absorbable (e.g., they mainly remain in the gut), and are also used for small intestinal bacterial overgrowth (SIBO). Additional examples of antibiotics include but are not limited to daptomycin, metronidozole, and linozelid, in which there has been a case study of AIDS patient with C. innocuum infection that was eradicated by these antibiotics.

In various embodiments, the antibiotic is not vancomycin when the bacterial strain is vancomycin-resistant. For example, some C. innocuum strains are vancomycin-resistant.

In various embodiments, the fibrosis treated is ileal fibrosis.

In various embodiments, the fibrosis is pulmonary fibrosis, liver fibrosis, heart fibrosis, mediastinal fibrosis, retroperitoneal fibrosis, skin fibrosis, adipose fibrosis, or scleroderma.

While not wishing to be bound by any particular theory, there is a growing body of evidence of gut connections to other organs in the body such as the gut-brain axis, gut-lung axis, etc. The gut is connected to all these other organs through the blood and lymphatics, which are modes by which bacteria or their metabolites can travel. The bacteria identified herein can be eliciting their effects by direct contact with other cell types, or by producing metabolites that interact with other cell types. Accordingly, the fibrosis is not limited to ileal fibrosis.

In various embodiments, the subject has inflammatory bowel disease. In particular embodiments, the subject has Crohn's disease. In other embodiments, the subject does not have ulcerative colitis.

In various embodiments, the agent is formulated into a pharmaceutical composition. Pharmaceutical compositions according to the invention may be formulated for delivery via any route of administration.

“Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

To understand factors that may be contributing to ileal CD sub-phenotypes, our study asked why MAT in the ileum undergoes such pronounced hyperplasticity at the sites of inflammation in CD. The so-called creeping fat morphology was a mystery and yet is consistently observed in CD and absent in UC. A possibility is that IBD intestinal barrier integrity is compromised facilitating the translocation of luminal and mucosal bacteria to MAT. This translocation is either exclusive to CD, or the types of microbiota translocating in CD vs. UC are different and therefore drive different phenotypes. We asked this question in two ways: 1) a within-patient comparison of involved and uninvolved resected intestinal tissue and MAT in 9 ileal CD and 9 UC patients; and 2) a between-disease comparison of the same paired samples between CD and UC.

Despite the latter being a comparison of small intestinal to colonic disease, we assessed them as an additional intestinal inflammation in the absence of CrF.

First, we found that bacteria and fungi translocate to MAT in both involved and uninvolved tissues, and in both CD and UC. V4 16s rRNA and ITS 1-2 and 3-4 sequencing of paired intestine and associated MAT or CrF revealed the presence of bacterial and fungal DNA in MAT that were also present in the intestine. While at a lower abundance and diversity in MAT, all sequences were also found in the luminal mucosa strongly indicating mucosal origin. Some bacterial sequences, but not fungi, were also found in abdominal wall adipose tissue (AAT), collected as an additional control, and were also of mucosal origin. This indicates bacteria can translocate to distant adipose sites, but success may be a function of distance (FIG. 1C, shown for a representative CD patient).

Second, we found that these organisms are alive in MAT. Our cultivation protocol utilized 20 standard and in-house developed media in anaerobic and aerobic conditions to systematically and maximally isolate bacteria and fungi from complex communities. Pure isolates from MAT and CrF were successfully grown and identified by full length 16s rRNA gene sequencing. We ensured there were no mesenteric lymph nodes (MLN) within or near our sampling sites. We separately isolated MLNs for cultivation when available and surprisingly never identified living organisms despite detecting bacterial sequences. It is possible that bacteria were simply dead or phagocytosed by antigen-presenting cells. Interestingly, there were several bacteria that were only found in CD. Most intriguing is Clostridium innocuum, which was present in both MAT and CrF in 6/9 CD patients. This is a highly motile, vancomycin-resistant bacterium. While it translocates in both CrF and uninvolved MAT, these appear to be different strains. The presence of cultivable C. innoccum from the colonic mucosa but complete absence from colonic MAT indicates its translocation may be a true phenomenon of ileal CD. C. innocuum therefore was further studied, including mechanistic exploration into its influence on ileal fibrosis and recurrence. The additional five bacteria exclusive to CD, and any emergent microorganisms is also systematically screened. There were no fungal species found exclusive to CD, however, the incidence of fungal translocation to MAT was greater in CD patients than UC.

Third, we found that these are not contaminants. Determining what is native vs. environmental is often the most challenging aspect of translocation studies. Single-cell RNA sequencing (scRNA-seq) is a useful tool for determining true biological response to presence of microbiota. Single-cell gene expression profiles from MAT and CrF macrophages, neutrophils, and B cells revealed upregulation of numerous anti-microbial genes for lysozyme, TLR4, TLR2, S100A9, and MEFV, for example, and displayed phenotypic subpopulations that are characteristically driven by the presence of pathogens. These expression profiles require chronic exposure and cannot be explained by exposure to bacteria at time of surgery. Despite aseptic technique and immediate transport from operating room to the sterile hood, presence of obvious contaminants, such as Staphylococcus species, were occasionally isolated and eliminated from our analyses and culture collection. However, these only occurred in 2/10 samples. Taken together, the data indicates ileal CD MAT, and CrF specifically, harbors a unique profile of translocated bacteria.

Fourth, we found that MAT from CD and UC have distinct cellular phenotypes defined for the first time at single-cell resolution. Single-cell RNA sequencing was performed on isolated stromal vascular cells (SVC) from CrF and paired MAT in three CD patients. Involved and uninvolved MAT from two UC patients were analyzed as an additional control. To our knowledge, this represents the first characterization of MAT and specifically CrF at single-cell resolution. Clustering was based on gene expression profiles from 1,200 cells per sample, and ˜200 genes per cell using the publicly available R package Seurat. Cells were encapsulated and barcoded using the Bio Rad ddSeq platform. Quality filtering and tertiary analyses were performed by collaborators at Harvard T.C. Chan School of Public Health. We identified cell clusters manually through knowledge of known cellular markers and predicted cell populations, combined with publicly available tools for identifying cell clusters based on gene expression data. The combined MAT profile for CD and UC and reveals nine distinct cellular populations in order from most to least abundant: 0-mesenchymal stem cells (MSC)/pre-adipocytes (PA), 1-macrophages (Mac), 2-T cells, 3-fibroblasts, 4-neutrophils, 5-B cells, 6-dendritic cells (DC), 7-endothelial cells (EndoC)/pericytes, 8-NK cells, 9-mast cells. Many of these cell types, such as macrophages, are well characterized in adipose tissue, while others such as DCs, B cells, NK cells and MSCs are poorly understood. When the t-SNE is delineated by disease, it becomes clear that MAT in CD vs UC have different cellular compositions. T cells and EndoC's are overrepresented in CD, for example, while neutrophils, NK cells and mast cells appear in abundance in UC-derived samples. In many ways this reflects the cellular immune patterns observed in the intestinal tissue, therefore indicating that the peri-intestinal tissue, and specifically MAT, is intimately interacting with the intestines during the course of disease.

Fifth, we found that CrF is characterized by a tenuous relationship between pro- and anti-inflammatory cells and hyperactive tissue remodeling. The single-cell data can be further interrogated to determine whether there are cellular distinctions between CrF and uninvolved MAT in CD. The combined cells from CD MAT and CrF show seven distinct cell types: 0-Fibroblasts, 1-T cells, 2-Adipose-derived stem cells (ADSCs), 3-B cells, 4-Macrophages, 5-Mesenchymal stem cells (MSCs), 6-Endothelial cells, 7-Myofibroblasts. When annotated by CrF or MAT, differential gene expression analysis comparing CrF to MAT revealed potential cellular mechanisms of fibrosis in CrF. The dominant antigen-presenting cell in u-MAT and CrF were macrophages, which, among the top 20 highly expressed genes, were upregulated for anti-microbial or microbial sensing genes such as lysozyme, complement factors C1QA and C5AR1, and toll-like receptor 2. These expression profiles are indicative of chronic exposure to microbial antigen rather than transient exposure at time of surgery (i.e. contamination). These data reinforce that the live microorganisms detected are not contaminants. Furthermore, the MAT of CD patients is transcriptionally upregulated for collagen production in five of the eight cell types detected and this was not seen in UC MAT. Interestingly, but not unexpectedly, inter-individual differences in cellular distribution across clusters was observed between patient specimens. However, each cell type was represented in each patient. Also striking, was the exclusive expression of TL1A on CrF macrophages (p=10⁻⁸). This was not seen in uninvolved MAT or UC MAT. TL1A is a member of the TNF superfamily and its expression is increased in the mucosa of inflammatory bowel disease patients. Moreover, a subset of CD patients with the risk TL1A haplotype is associated with elevated TL1A expression and a more severe disease course.

Furthermore, the mac population (cluster 2) expresses both M1 and M2-like phenotypes, but is clearly skewed toward TGF-β and VEGF producing M2 cells which have been described as potent activators of myofibroblasts. Studies have shown that while M2 macs initially serve a regulatory and anti-inflammatory role to promote wound healing and repair, when a lesion is persistent M2 macs can become pro-fibrotic and stimulate fibroblast proliferation. The most differentially expressed gene in the CrF fibroblast cluster is the downregulation of DKK-1, a potent inhibitor of Wnt signaling, and upregulation of WISP-1, a canonical Wnt activator. Studies show that TGF-β stimulates canonical Wnt signaling via downregulation of DKK-1 and promotes fibrosis by fibroblasts. This pathway is evident in CrF and may be responsible for the fibrotic cascade differentiating CrF from uninvolved MAT. These data inform our hypothesis that microbial translocation to MAT drives an initial pro-inflammatory response that remains evident to a degree by the presence of activated neutrophils and M1 macrophages. However, the overall immune profile in CrF is one of an adapted immune response attempting to regulate inflammation in the presence of unresolved ileal injury. This prolonged signal to protect and heal, ultimately leads to uncontrolled collagen matrix deposition not only from fibroblasts, but from the immune cells as well. In genetically susceptible individuals, we speculate this prolonged cascade remains uncontrolled and leads to fibrosis.

Sixth, we found that increased intestinal permeability in inflamed ileum facilitates translocation of microbiota but does not promote systemic inflammation. Disruption of intestinal tight junctions and related proteins leads to intestinal permeability facilitating the passage of luminal contents to neighboring tissues and systemic circulation. Our study detected a significant microbial load in the MAT. Considering the size of fungi alone, up to 30 um, indicates significant permeability.

Gene expression of e-cadherin, zonulin-1, claudins 3,4,7 and 12, tricellulin, JAM-A, muc-1, and trefoil factor -1 was measured by qPCR from full thickness biopsies from involved and uninvolved ileum and colon in CD and UC resections. Significant difference between groups was only detected in e-cadherin, claudin-3, and claudin-7. These data show that involved ileum is more permeable than its adjacent uninvolved tissue, which may play a role in bi-directional signaling to CrF. Permeability appears greatest in UC compared to control, however, the difference in permeability between CD and UC is not significant. This is meaningful when considering plasma lipopolysaccharide binding protein (LBP) is significantly greater in UC patients compared to CD. This may indicate CrF, while potentially exacerbating the fibrotic milieu, serves as a biocontainment barrier for translocated microbiota. Together these preliminary data provide evidence for the presence of microbial translocation from the intestine to mesenteric fat, and for the first time identifies a collection of six live bacteria unique to CrF. We have also, for the first time, identified fungal translocation to MAT and have successfully sequenced and isolated these organisms. While we have not yet identified fungal species exclusive to CrF, we cannot exclude their influence on the microbial milieu of CrF, and will continue to investigate these species alongside the bacterial community. We have also, for the first time, characterized the MAT at single-cell resolution, and specifically, have curated and extensive gene expression profile of every cell type in CrF.

### Example 2. Determination of Whether Patients' Polygenic Risk Score is Associated with Inflammation, Fibrosis, and Blooms of Certain Bacteria/Fungi in Ileal CD

Treatment strategies for Inflammatory bowel diseases (IBD) have historically been determined by a diagnosis of CD or ulcerative colitis (UC). However, there is growing evidence that IBD is a spectrum of disease within these binary classifications requiring a new classification. This indicates that treatment plans can be meaningfully improved through more comprehensive classification of disease incorporating both genotype and phenotype. Recently, the International Inflammatory Bowel Diseases Consortium conducted the largest IBD genotype-phenotype study to-date in 29,838 patients, and generated polygenic genetic risk scores by summarizing the total load of genetic risk for a particular phenotype and found that the strongest association was between genetic risk score and disease location. The study determined that ileal CD is as genetically distinct from colonic CD as it is to UC, and has the shortest time from diagnosis to complicated disease or surgical intervention. These disease complications in ileal CD, such as uncontrolled inflammation and fibrotic strictures, represent more than 50% of the cases, and represent an important sub-phenotype for characterization, and an area of unmet medical need given that the majority of these patients will require surgery at some point in their life. Propensity for inflammatory and stricturing disease is likely explained by a combination of genetic variation and the ileal microenvironment including the microbiome.

The general strategy is to generate a large and diverse dataset of host and microbiome readouts from patients with ileal CD (1a), and to combine these with patients' calculated polygenic risk scores (1b), to determine whether associations exist that can predict likelihood for developing fibrosis.

### 1a: Curate a Multi-'Omic Dataset

We will collect the following samples from 40 ileal CD patients at time of resection: 1) involved ileum and attached CrF, 2) adjacent uninvolved ileum and attached MAT, 3) whole blood. From these tissues we will generate microbiome sequence data from the mucosal-associated and adipose communities using ITS and 16s rRNA gene sequencing. A subset of microbial samples (n=10 patients, 40 samples total) will be analyzed by whole genome sequencing. Single-cell whole transcriptome data will be generated from the intraepithelial lymphocytes (IEC) and lamina propria (LP) of involved and uninvolved ileum and paired CrF or MAT from six patients selected based on highest and lowest polygenic risk score generated in Aim 1b. We will generate data at a resolution of 3,000 cells per sample using the 10× Chromium system, which we have in-house in our Bioinformatics and Functional Genomics (BFG) core. Our lab developed a protocol for isolation and freezing single cell suspensions for scRNA-seq, which is being utilized by Bio Rad Genomics as a companion protocol for the ddSeq platform. The flexibility to reliably store samples for later analysis allows maximal utilization of sample collection on the day of surgery, and prevents sample handling errors due to time pressures on a given day. This will generate gene expression data from 15,000 cells each from involved and uninvolved ileum, and CrF and MAT.

Computational and bioinformatics support will be provided by our collaborators, Shannan Ho-Sui and Michael Steinbaugh at Harvard T.C. Chan School of Public Health, whom we have previously worked with to analyze the ScRNA-seq data from our preliminary data set. We additionally have in-house bioinformatics support from the BFG, and we have a post-doctoral fellow in our group concurrently being trained in these analyses. Tight junction gene expression from full thickness ileal segments will be analyzed and LBP assays will be performed as described in preliminary data, as well as C-reactive protein. The Cedars MIRIAD biobank additionally performs the following clinical assays on each patient which we will include in our dataset: ASCA, ANCA, OmpC, and CBir.

### 1b: Determining Association with Polygenic Risk Score

Disease polygenic risk scores (PRS) will be calculated as a weighted sum of the number of risk alleles carried by each individual (0, 1, or 2) at known disease loci, with weights proportional to the effect estimates from the previously published large-scale association studies. R package Mangrove will be utilized in the PRS calculation. Association between PRS, clinical phenotypes, and 'omics data will be evaluated within the Generalised Linear Model (GLM) framework. Cox Proportional Regression will also be used when analyzing survival data. Principal Components (PCs) from population stratification analysis will be incorporated when applicable to control for potential confounding effects.

### Power Analysis

We will utilize our in-house metagenomic sequencing pipeline to align the bacterial/fungal sequences and call for taxa. Assuming that there are there are 300 taxa available for downstream analysis after preprocessing the data, we will first normalize the data with arcsine square root transformation, then the power is estimated with two-side one sample Student's t-test. Given the statistical significance level of 0.00017 (0.05/300) with Bonferroni justification for the multiplicity problem, the sample size of 40 of paired samples will have 80% power to detect the effect size of 0.75. The Power for RNA-seq was estimated with the s-sizeRNA R package. Given a total of 10,000 mRNA genes, proportion of differentially expressed (DE) genes across different clinical conditions of 20%, an average read count for each gene in control group of 10, false discovery rate of 0.05, sample size of 40 in each group achieves 98% and 87% powers to detect the fold change 2 with the corresponding dispersion parameters of 0.3 and 0.4, respectively.

We expect to find an association among our phenotypic readouts because our preliminary data on a much smaller cohort already indicates a relationship exists between the gut microbiota, MAT and fibrosis. There will be an additional layer of granularity by single-cell sequencing the LP in paired tissues from the same patient.

An unknown in the field of single-cell genomics is the degree of inter-individual heterogeneity. While cell populations do not likely change from one individual to the next within a specific disease, their gene expression profiles may. We therefore will primarily compare samples within each individual, using the uninvolved samples at the individual control and relative comparator. Genome-wide studies have remained elusive in their predictive capabilities, therefore we expect polygenic risk scores which take into account host phenotypes, to inform significant associations with the microbiome. Alternative approaches to this would be to create an epithelial integrity score which would be a polygenic risk score based on genetic risk variants for IBD that targets genes associated with barrier integrity.

### Example 3. Define the Microbial Contribution to Pro-Inflammatory and Pro-Fibrotic Responses and Disease Recurrence

For over a decade, the gut microbiome has been identified as a trigger in IBD in genetically susceptible individuals. However, IBD consists of many sub-phenotypes and determining in which of these the microbiome may be most influential requires more human studies. CD fibrosis represents a sub-phenotype of CD affecting 30-50% of patients. Seventy-five percent of these individuals will have surgery for fibrotic strictures (1) and most of these will develop recurrent strictures requiring surgery. While therapy exist to manage CD inflammation, none exist to prevent or treat fibrosis. Fibrosis is defined as the unregulated deposition of extracellular matrix (ECM) proteins. Production of ECM proteins is often a wound-healing response, however aberrant signals can lead to continual activation. Our data shows that CrF is a fibrotic tissue compared to adjacent uninvolved MAT, and is likely driven by underlying inflammation in the ileum and bacterial translocation into MAT. The cellular milieu of CrF represents acute-phase immune cells as well as adaptive immune cells such as plasma cells and M2 macrophages. Interestingly, this adaptation to the presence of microbiota, appears to drive constituent activation of ECM-producing fibroblasts. A critical decision point of inter-individual variation in this response may lie in how the host immune cells respond to the selected microbiota. We believe that CrF may be both modeling and contributing to the fibrotic events occurring in the ileum, and that these events are microbially driven. Therefore, a fixed background of genetic susceptibility combined with persistent colonization of certain microbiota may predict who will develop fibrosis and recurrent disease.

A focus will be on predictive measures of post-operative recurrence. This will be achieved through characterizing the cellular phenotypes of post-operative biopsies (2a) and screening candidate immunogenic and fibrogenic microbiota against patient's own immune cells (2b/2c).

### 2a: Characterization of Post-Operative Cellular Milieu by Single-Cell RNA Sequencing

We will collect 6-month post-operative biopsies as outlined in the Cedars-Sinai initiated, NIDDK approved Inflammatory Bowel Disease Genetics Consortium (IBDGC) study. This study is ongoing, and currently collecting post-operative biopsies, stool, blood, and diet records from patients who have undergone ileal resection for CD. The 40 patients proposed in Aim 1 will also be enrolled in this study. The study SOP specifies collection of post-operative biopsies at 6, 24, and 48 months post-ileal resection (Table 1). Given the timeline of the current proposal we are only proposing collection of the 6-month time point, but we intend to separately analyze the 24 and 48-month time points in a later study. All sample handling will be in accordance with the study SOP. Briefly, two biopsies will be endoscopically collected for RNA analysis. We will pool these samples and isolate IEC and LP single-cell suspensions and process for scRNA-seq as described in Aim 1a. We will compare the cellular gene expression profiles from these post-operative samples to the profiles generated at time of resection. We will specifically be looking for whether there is differential expression in collagen matrix and bacterial responsive genes between resection and 6-months post-op, and whether this is correlated to the individual's polygenic risk score. We will also collect two separate biopsies for 16s rRNA sequencing as well as stool samples to characterize the mucosal and luminal microbiota post-op and compare to the profile at time of surgery. This will allow us to determine whether persistence of our candidate microbiota in combination with persistent cellular phenotypes denote a central characteristic of individuals genetically susceptible to fibrosis and recurrence.

TABLE 1
NIDDK IBDGC Ileal Post-Op Study Timeline

| | |
|---|---|
| Phase 1 | Phase 2 |

Events/Samples

| | | | | | | | | |
|---|---|---|---|---|---|---|---|---|
| Enrollment¹ | X | X | | | | | | |
| Blood | X | X² | | | (X²) | | (X²) | |
| Diet | X | X | X | X | (X²) | | (X²) | |
| Endoscopies | | X | | | (X²) | | (Xx²) | |
| Stool² | | X | | | (X) | | (X) | |
| Status updates | | | | X | X | X | X | X |
| Time after surgery | 3 | 6 | 9 | 1 | 2 | 3 | 4 | 5 |
| | mos | mos | mos | yr | yrs | yrs | yrs | yrs |

### 2b: Screening Candidate Microbiota for Immunogenic and Fibrogenic Potential

This aim seeks to delineate at a mechanistic level how an individual bacterium interacts with a patient's immune cells and what response it elicits. Our preliminary scRNA-seq data reveals that hallmarks of CrF include an over-representation of TGF-β and VEGF producing M2-like macrophages, enhanced Wnt signaling in fibroblasts, and pan-downregulation of heat shock proteins in all immune cells, but primarily B cells. We will determine whether exposure of these cells to bacterial antigen from our candidate bacteria can alone stimulate these pathways. This will allow us to determine whether a specific microbiota can be targeted for intervention. We have identified C. innocuum as our primary candidate bacterium to screen for immune stimulating/differentiating and fibrogenic potential. This organism was present in 6/9 CrF samples and no UC MAT samples despite detection in both ileal and colonic mucosa. C. innocuum was also the only bacterium, other than Clostridium perfringens, which was ubiquitously detected, that displayed exceptional motility when subjected to a motility stab test. Most bacteria detected in the CrF were not motile to any detectible degree, indicating motility is not a requirement for translocation, however, C. innocuum's exceptional motility may be a property that elicits response in host cells. To this end, we will isolate peripheral blood mononuclear cells (PBMCs) from each patient (n=40) and culture them with C. innocuum lysate. We will also test lysate from five additional candidate bacteria found exclusively, but not consistently, in CD CrF: Parabacteroides distasonis, Erysipelatoclostridium ramosum, Clostridium symbiosum, and Veillonella parvula. We also tested lysate from Ruminococcus gnavus. We will repeat this assay with tissue resident macrophages isolated from CrF, and ileum from surgical resections. Cells and bacterial lysate will be co-cultured for 72 h, with samples of supernatant collected at 6, 12, 14, 48, and 72 h for cytokine analysis by ELISA. Based on findings from our preliminary data, we will also measure TGF-β and VEGF. Cells will be harvested and analyzed by qPCR for gene expression of collagen matrix genes. In parallel, we will isolate adipose-derived stem cells/fibroblasts (ASCs) from the SVC fraction of CrF and uninvolved MAT. These cells will be co-cultured with supernatant derived from the stimulated PBMCs and macrophages to determine whether macrophage stimulation of fibroblasts promotes upregulation of Wnt signaling and ECM gene expression. While the ASC's from CrF may already be stimulated and therefore exhibit a blunted response to additional stimuli, we will test whether we can create a CrF-like gene expression profile in uninvolved MAT through in vitro stimulation.

We have in vitro data supporting that bacterial candidate, Clostridium innocuum, can polarize patients' macrophages to TGF-β producing M2 cells, which serve a key role in cell proliferation and fibrosis. When we exposed CrF tissue explants that contain ASCs to various bacteria, we found that Clostridium innocuum, Ruminococcus gnavus and Erysipelatoclostridium ramosum increased the expression of ECM genes by several folds compared to other treatments. Moreover, tissues that were exposed to Clostridium innocuum had lower expression of DKK-1, and downregulation of DKK-1 is implicated in activation of Wnt signaling and promotion of fibrosis. Data here are in line with the observation from our single-cell RNA sequencing above. Notably, a bacterial cocktail without the candidate species induced very little changes in ECM or DKK-1 expression. This supports our hypothesis that bacterial candidates associated with CrF have high fibrogenic potential compared to other species.

### 2c: Test Whether Lymphoblastoid Cells from Patients with Recurrent Fibrosis are Differentially Stimulated by Bacterially-Stimulated APCs Compared to Healthy Controls

B cells will be exposed to bacterially-stimulated APC supernatant from 2b. B cells represented a very interesting cell population in our scRNA-seq dataset because of its maturation profile, and pan-downregulation of heat shock proteins. We will utilize the NIDDK Rutgers Cell and DNA Repository to request lymphoblastoid cell lines derived from CD patients who have undergone surgery for fibrotic strictures (n=20) and from healthy controls (n=20). We will request 20 cell lines per year over two years. While in 2b each cell patient's cells are stimulated with each patient's own bacteria. However, for this aim the data will be difficult to interpret if we use different supernatants on each cell line. Therefore, we will create a representative supernatant “mastermix” based on cytokine and gene expression profiles we feel are most representative of our population. This single mastermix, constituting and average from multiple patients, will be used for incubation with the lymphoblastoid cells. We will measure expression of heat shock proteins(HSP) 70 and 90, as well as HSP family gene DNAJB1. We will also determine expression of ECM genes, as our preliminary data revealed all immune cells in CrF had upregulated expression of ECM genes. Differential expression will be assessed between the CD-derived lines and healthy controls, but we will also analyze unstimulated cells in both CD and healthy as a basal comparison.

These lymphoblastoid lines were derived from patients enrolled at IBDGC sites. Clinical information will be obtained in advance from the IBDGC sites as to which lymphoblastoid lines come from donors who underwent surgery for fibrotic strictures, and only choose those individuals to analyze in the treatment group. Data regarding whether each patient developed recurrent fibrosis and the time to recurrence will also be requested.

We anticipate that the cellular phenotype and microbiota composition profile in post-op ileal biopsies will be similar to the profile at time of surgery in individuals with a strong polygenic risk score for ileal fibrosis. If there are not enough high-quality cells from biopsies to perform scRNA-seq, we will perform bulk RNA seq on these samples. We will have enough ileal tissue at time of resection to perform scRNA-seq at for this time point, and this gene expression profile can then be used to infer the cellular origin of expressed genes in biopsy samples. We have extensive experience performing immunogenicity assays with bacterial lysates and primary immune cells and anticipate one or more of our bacterial candidates to elicit a response in isolated macrophages. In this case we will rely on qPCR or bulk RNA seq methods to recreate our known expression profiles.

### Example 4. Determination of Whether a Patient's Translocated Microbial Consortium, or Specific Organisms, are Capable of Driving Intestinal Inflammation and Fibrosis in a Germ-free, Genetically Susceptible Model of Fibrosis

Determining the characteristics and behaviors of individual microbiota presents a challenging task when the organism is a member of the native gut microbiome presenting a complex community. Identification of the organism in large disease populations in humans gives a sense of its potential clinical relevance. Isolating the organism and testing in vitro allows one to manipulate the environment in a very controlled, albeit artificial way. This allows us to answer very specific questions. However, introducing the microbiota into germ-free animals represents the combination of an in vivo, self-propagating system while maintaining control over variables. Furthermore, this model will allow us to test Koch's postulates which establishes the series of tests one must perform to determine if an organism is causative (or strongly implicated). The postulates state that in order to determine whether a microorganism is a potentially driving factor in disease, the organism must 1) be present in every case of the disease (in our case present in 6/9 CD CrF, and absent in UC MAT and AAT); 2) isolated and grown in pure culture (we have cultures from each patient), 3) when re-introduced into a healthy genetically susceptible host the organism must re-create the disease; and 4) when re-isolated from the diseased host it must be identified as the original strain.

We will determine whether C. innocuum or any of our CrF-specific microbiota are capable of driving fibrosis in a genetically susceptible host and to gain insight into the early progression and time course of fibrosis (3a), while also assessing whether these microbiota retain their ability to translocate and whether this precedes fibrosis (3b).

### 3a: Study the Effect of Candidate Microbiota on the Time Course of Fibrosis in Genetically Susceptible Gnotobiotic Mice

The TIL1A transgenic mouse is an established mouse model of spontaneous ileal fibrosis that mimics the fibrotic phenotype seen in Crohn's disease. Therefore, we believe this is a well-suited model in which to test the influence of our selected patient-derived microbiota on disease course.

Germ-free and gnotobiotic animals are essential research models for microbiome-based studies. While they harbor some inherent physiological differences compared to conventionally-raised animals, such as decreased intestinal mucus secretion, and hyperphagia, genetically susceptible disease models tend to be resistant to developing disease until microbiota are introduced. This offers an opportunity to observe the differential time course of disease in the presence of different microbiota. Here we will utilize germ-free TL1A overexpressing mice (TL1A tg) and wild-type (WT) controls that have been re-derived germ-free at the University of North Carolina Gnotobiotic Resource Core. We will request three breeding pairs each of TL1A tg and WT to achieve a total of 20 mice for inclusion in our study. We will use both males and females, as no gender difference has been observed in fibrosis development in these mice. These mice will be housed in our gnotobiotic animal facility and maintained by my trained research staff. Mice will be raised in dedicated breeding isolators, and split between two isolators in the unlikely event of a contamination we do not lose the entire population. At 6-8 weeks old, mice will be transferred to experimental isolators dedicated to each of the selected microbiota. Mice will be gavaged with either C. innocuum or a mixed consortium of our candidate and maintained on autoclaved gnotobiotic mouse diet and. TL1A-mediated fibrosis becomes apparent at around 16 weeks of age in conventionally-raised mice, so we will use this as our benchmark for an endpoint.

Mice will be divided across the following groups of n=20 each: 1) TL1A tg+C. innocuum, 2) TL1A tg+mixed microbial consortia, 3) TL1A WT+C. innocuum, and 4) TL1A WT+mixed microbial consortia. Within each of these cohorts, five mice will be terminated at T=4, T=8, T=12, and T=16 weeks post-gavage. We will be examining whether either C. innocuum alone, or as a member of a consortia, increases the penetrance, severity, or shortens the time to fibrosis. Fibrosis will be assessed qualitatively, and histologically, as well as by qPCR of ECM genes. We will additionally collect stool samples from each mouse every two weeks to create a secondary time course of microbial changes. We will re-isolate the introduced microbiota from the ileal mucosa at each time point and submit for full length 16s sequencing to ensure both colonization and strain persistence over time.

### 3b: Determine if Microbiota Retain the Ability to Translocate to MAT and Stimulate Fibrotic Gene Expression

We will determine whether C. innocuum or members of the microbial consortia retain their ability to translocate to the MAT during the time course of fibrosis. While TL1A tg and WT mice do not develop CrF, we have shown that CrF is not required for bacterial translocation in general. We believe that the fibrotic ileal environment facilitates passage of microbiota to MAT, therefore we expect to find these microbiota in the MAT. We will harvest MAT at each time point and analyze this tissue by bulk RNA seq for downregulation of heat shock proteins, and upregulation of TGF-β and VEGF, Wnt pathway, and ECM genes. We will also terminate a subset of five non-colonized TL1A tg and WT mice each for above analyses. This analysis will allow us to determine whether these microbiota alone are capable of driving the cellular phenotype of fibrosis we have previously defined.

The power is estimated with two-sample Student's t-test. Given the statistical significance level of 0.05, the number of 5 mice will have 80% power to detect the effect size of 1.7. Because of the repeated measures of multiple time points, the power will be even larger than estimated.

We anticipate that C. innocuum and the microbial consortia will successfully colonize and induce measurable change in the pathophysiology of ileal fibrosis in these mice. We will assess by regular stool sampling at bi-weekly intervals and analysis of the mucosal associated community at the termination time points. If we find lack of persistence, we will re-gavage the mice at intervals determined by our analysis. However, we will attempt to space these gavages as far apart as possible to reduce animal stress. Contamination of germ-free breeding isolator is always a risk. To ensure ongoing sterility during the course of this study we will monitor isolators as outlined in “Facilities: Animal Resources” and “Authentication of Key Resources”. Briefly, we analyze stool by cultivation and PCR weekly, and submit random blood samples twice a year for serology testing for pathogens. While we speculate that we can reproduce the gene expression profiles we identified in human samples, we recognize that rodent models do not always behave the same way. We are realistic in these challenges, so rather than take a targeted qPCR-based approach looking only for our pre-identified genes and pathways of interest, we have proposed utilizing bulk RNA seq so that we may get a more comprehensive expression profile. This will allow us to both interrogate our selected pathways, but also make conclusion about analogous pathways.

We are studying an innovative combination of polygenic risk score, microbiome, and cellular gene expression of both the intestinal and peri-intestinal milieu to characterize ileal fibrosis in CD with unprecedented granularity.

### Example 5. Translocation of Viable Gut Microbiota to Mesenteric Adipose Drives Formation of Creeping Fat in Humans

### 16S Sequencing Reveals Bacterial and Fungal Signatures in Creeping Fat That Are of Gut Origin

Paired involved and uninvolved ileal segments with attached CrF and adjacent normal MAT, and blood, for a total of five regional sites per patient, were obtained from 11 patients undergoing surgical resections due to complications from CD. In addition to the paired within patient samples, we collected the same five regions from UC patients (n=13) as controls who exhibit intestinal inflammation in the absence of creeping fat. These samples were placed through a systematic workflow of sample processing and analysis (FIG. 1E). Patient metadata including clinical characteristics, medication use, family history, social history and demographic information of this study cohort are detailed in Table 2.

TABLE 2
Patient metadata.

| | | Crohn's | Ulcerative |
|---|---|---|---|
| | | disease | colitis |
| | | (n = 11) | (n = 13) |
| Gender | Male | 7 | 7 |
| Race | European | 3 | 3 |
| | ancestry/Jewish | | |
| | European | 6 | 7 |
| | ancestry | | |
| | Asian | 1 | 0 |
| | Pacific | 1 | 0 |
| | Islander | | |
| | Hispanic | 0 | 1 |
| | Other | 0 | 2 |
| Family history | Yes | 1 | 5 |
| of IBD | No | 10 | 8 |
| Median age (min.- | | 49 (18-75) | 37 (20-72) |
| max.) | | | |
| Median age at | | 35 (7-60) | 26 (11-60) |
| diagnosis (min.-max.) | | | |
| Median duration | | 15 (9-19) | 5 (1-25) |
| of disease (min.-max.) | | | |
| Previous | Yes | 7 | 0 |
| IBD surgery | No | 4 | 13 |
| Montreal | A1 | 3 | |
| classification | A2 | 5 | |
| CD† | A3 | 3 | |
| | B1 | 0 | |
| | B2 | 11 | |
| | B3 | 4 | |
| | L1 | 2 | |
| | L2 | 0 | |
| | L3 | 9 | |
| | p | 1 | |
| UC | E1 | | 0 |
| | E2 | | 1 |
| | E3 | | 12 |
| Indication for | Strictures | 7 | 0 |
| surgery | Strictures | 2 | 0 |
| | w/fistulae | | |
| | Medically | 0 | 13 |
| | refractory | | |
| | Obstruction | 2 | 0 |
| Smoker | Never | 5 | 10 |
| | Current | 0 | 0 |
| | Ex-smoker | 5 | 3 |
| | N/A | 1 | 0 |
| Medications* | Aminosalicylates | 1 | 7 |
| | Corticosteroids | 3 | 8 |
| | Immunomodulators | 7 | 6 |
| | Biologics | 9 | 12 |

†Patients may be classified in more than one “B” category
*Patients could be on more than one class of medication
Montreal Classification:
CD (A1-below 16 y; A2-between 17 and 40 y; A3-above 40 y; L1-ileal; L2-colonic; L3-ileocolonic; B1-non-stricturing, non-penetrating; B2-stricturing; B3-penetrating; p-periaanal disease modifier) and
UC (E1-Ulcerative proctitis; E2-Left sided UC (distal UC); E3-Extensive UC (pancoloitis)).

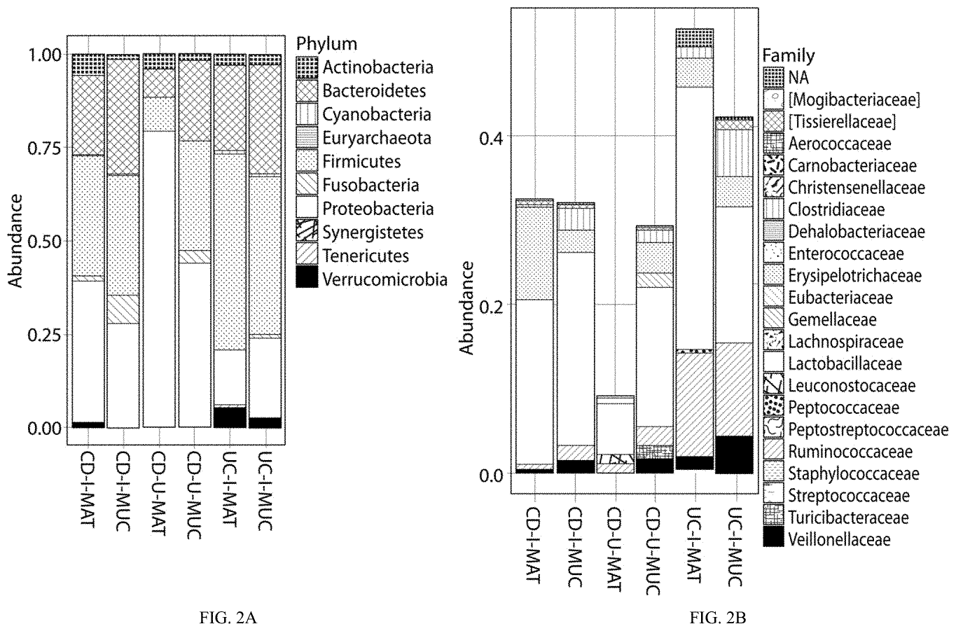

We performed high-throughput 16S rRNA sequencing to first assess whether bacterial DNA was detectable in MAT of IBD patients, and if so, how the distribution compared to the mucosal compartment. Irrespective of CD or UC disease, bacterial DNA was isolated from adipose specimens but at a lower alpha diversity compared to their mucosal counterparts (FIG. 2A). Mucosa from colonic UC resections was significantly higher in bacterial sequence variants than that of ileal CD. Expectedly, this reflects the bacterial richness of the colon compared to the small bowel. To determine if the bacterial footprint in extra-intestinal sites are native, translocated, or due to environmental contamination, we compared the taxa identified in the MAT (both paired specimens) to the mucosa and looked for overlapping taxa and outliers. Principal coordinate analysis using Bray-Curtis distance showed no unique clustering between the mucosa and MAT in either CD or UC resections indicating no significant distinction between the microbiota of the mucosa and adipose. To ensure luminal content contamination of MAT resulting from surgery is not a confounder, we vetted a detailed standard operating procedure in the operating room for sample collection which entails carefully suturing each end of the resected specimen to eliminate possible leakage of luminal content onto other sites. In the event that any luminal content contaminated adipose upon resection, or abscesses were identified, these samples were eliminated from analysis. Environmental exposure of the sample was also limited as specimens were transported directly to a sterile biosafety cabinet for processing in less than 20 minutes from time of resection, and MAT was always dissected first before removing the intestinal sutures. At the individual taxa level, MAT-derived bacteria were phylogenetically aligned to members of the mucosal microbiota in both CD and UC indicating that MAT does not represent a novel microbial niche, but rather translocation from the gut to MAT. Relative abundance data showed that CrF specimens were characterized by an expansion of Erysipelotrichaceae compared to their underlying ileal CD specimen and uninvolved MAT (FIG. 2A, 2B).

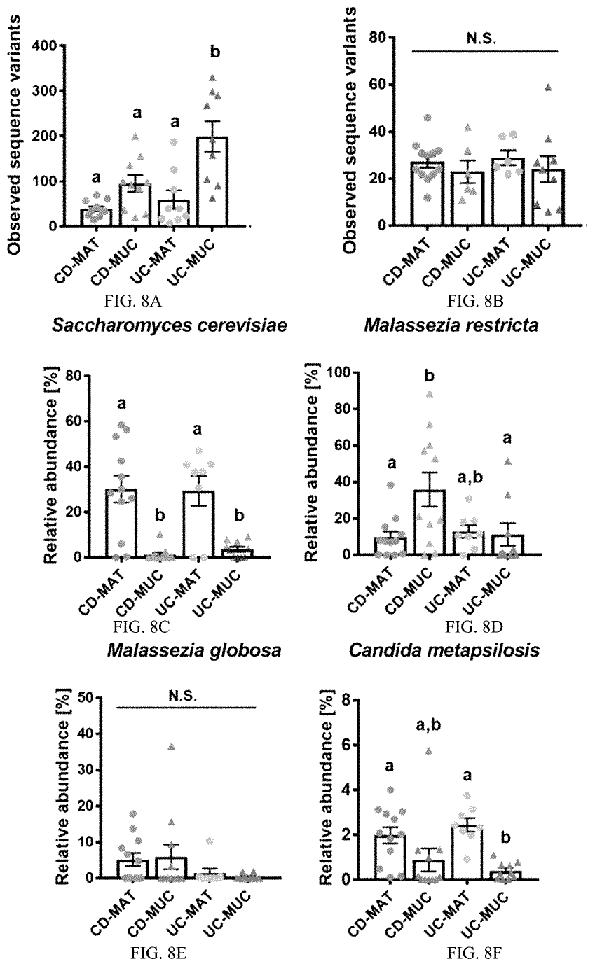

In addition to bacterial sequences, fungal DNA was also identified in all adipose and mucosal specimens from CD and UC resections. Unlike the 16S rRNA dataset which showed colonic mucosal specimens from UC had the greatest bacterial diversity (FIG. 8A), there was no significant difference in the number of observed ITS sequence variants between specimen location (FIG. 8B). Overall, fungal diversity in the gut is substantially lower than the bacterial counterpart and thus the intestinal and MAT-associated fungal communities were comprised of fewer types of organisms. Principal coordinate analysis revealed that fungal communities were largely separated by specimen location (MAT vs. mucosa) indicating that while there was no regional specificity in overall fungal diversity, the community structure reflects anatomical differences in this particular disease context and some species appear to markedly expand in MAT. In particular, the representation of Saccharomyces cerevisiae and Candida metapsilosis was significantly higher in MAT specimens irrespective of disease subtypes (FIG. 8C-8F). Relative abundances of Malassezia spp., on the other hand, showed CD specificity, particularly in the mucosa. Of the Malassezia, M. restricta in particular was significantly more prevalent in the mucosa of CD patients than that of UC. M. restricta was also detectable in the attached MAT but at a lower abundance. This organism is particularly interesting in the context of creeping fat because Malassezia spp. lack the genes for fatty acid synthesis and thus, their growth is dependent on the presence of long-chain fatty acids in the immediate environment. This increased abundance in the CrF-associated mucosa indicates the small bowel may experience a chronic influx of long-chain fatty acids as the mesenteric adipose depot expands.

### Creeping Fat is a Reservoir of Viable, Cultivable Organisms with Disease Specificity

While amplicon-based sequencing data is useful for providing a snapshot of the organisms present in a given sample, whether these identified organisms are passive bystanders dead upon arrival, or active inhabitants of a given niche cannot be reliably determined. We predicted that constituents of metabolically active organisms would have greater potential in shaping host physiology than degraded microbial cells, and may be able to better differentiate CrF from non-CrF MAT. Therefore, cultivation methods were used to assess the viable fraction of the MAT-associated communities at the time of sampling. All dissected mesenteric adipose depots from CD and UC were immediately transferred to the anaerobic chamber for processing. Eight different in-house modified media were used for cultivation which were created and optimized for supporting growth of the different classes of organisms we identified by sequencing. Bacterial colony forming units (CFUs) were recovered from the MAT of most cases except for two CD and four UC patients, despite identifying microbial sequences in those tissues. In total, we characterized 229 isolates which binned into 84 species (Table 3). Sixty-two of these species are obligate anaerobes whereas the remaining 22 are facultative anaerobes when tested in vitro. Of the facultative anaerobes, representatives from Staphylococcus and Streptococcus were isolated. While these genera typically associated with the skin and oral cavity, previous studies have shown that the expansion of these aerotolerant organisms aligned with active and severe IBD. When overlaying the cultivable bacteria (#=43) with those identified by 16S rRNA sequencing (#=91), only 41 species were detected by both methods, others were exclusively detected by sequencing or cultivation. For example, Akkermansia mucimphilia and Faecalibacterium prausnitzii were frequently detected by sequencing but not by cultivation. Yet, we validated that the culture media used in this study could support the growth of their type strains. This suggests only a fraction of the intestinal organisms can translocate to MAT and remain viable. In summary, 31% of the MAT-associated species from the sequencing data were viable at the time of sampling, and viable organisms were isolated in 18/23 patients. On the other hand, only two live fungal isolates were recovered from MAT, Candida albicans and Pseudozyma aphidis, and only in two CD patients. Both organisms were observed in their hyphal form, and while little is known about P. aphidis, several clinical reports have identified it as a human pathogen rather than a member of the commensal fungi. However, isolating specific fungi is notoriously challenging, and we cannot exclude the possibility that our growth media was not sufficient to support growth.

TABLE 3
List of all bacterial isolates from mesenteric adipose.
List of cultivable organism from mesenteric adipose tissue

| | |
|---|---|
| Actinomyces odontolyticus | Flavonifractor plautii |
| Alistipes finegoldii | Fusobacterium nucleatum |
| Arabia massiliensis 94% | Fusobacterium ulcerans |
| (Paraeggerthella 100% by RDP) | Gardnerella vaginalis |
| Bacteroides caccae | Gemella sanguinis |
| Bacteroides cellulosilyticus | Haemophilus parainflluenzae |
| Bacteroides fragilis | Hungatella hathewayi |
| Bacteroides ovatus | Klebsiella oxytoca |
| Bacteroides thetaiotaomicron | Klebsiella pneumoniae |
| Bacteroides uniformis | Klebsiella spp. |
| Bacteroides vulgatus | Lachnoclostridium sp. |
| Bifidobacterium bifidum | Lactobacillus gasseri |
| Bifidobacterium longum | Lactobacillus rhamnosus GG |
| Bifidobacterium pseudolongum | Massiliomicrobiota timonensis |
| Bilophila wadsworthia | Massiliomicrobiota timonensis |
| Blautia coccoides | Merdimonas faecis 95% |
| Blautia hansenii | (Unclassified Lachnospiraceae) |
| Blautia obeum | Morganella morganii |
| Blautia sp. KLE 1732 97% | Oscillibacter ruminantium |
| Cetobacterium somerae | Parabacteroides distasonis |
| Citrobacter freundii | Parabacteroides johnsonii |
| Clostridium baratii | Parabacteroides merdae |
| Clostridium bolteae | Parvimonas sp. |
| Clostridium botulinum | Phascolarctobacterium 100%; |
| Clostridium chauvoei | Phascolarctobacterium |
| Clostridium innocuum | succinatutens YIT 12067 91% |
| Clostridium perfringens | Proteus mirabilis |
| Clostridium spiroforme | Rothia mucilaginosa |
| Clostridium symbiosum | Ruminococcus gnavus |
| Collinsella aerofaciens | Ruminococcus torques |
| Desulfovibrio desulfuricans | Staphylococcus capitis |
| Dorealongicatena | Staphylococcus epidermidis |
| Eggerthellalenta | Staphylococcus haemolyticus |
| Eisenbergiella tayi | Staphylococcus saccharolyticus |
| Enterococcus faecalis | Streptococcus anginosus |
| Enterococcus gilvus | Streptococcus australis |
| Enterococcus hermanniensis | Streptococcus lutetiensis |
| Erysipelatoclostridium ramosum | Streptococcus parasanguinis |
| Erysipelotrichaceae 97% | Streptococcus sanguinis |
| Coprobacillus 90% | Tyzzerella nexilis |
| Escherichia/Shigella | Unclassified Enterococcus |
| Eubacterium oxidoreducens | Unclassified Erysipelotrochaceae |
| Eubacterium sp. Marseille | Veillonella atypica |
| (BLAST:98%)/Unclassified | Veillonella parvula |
| Lachnospiraceae (RDP: 100%) | |
| Faecalicatena contorta | |

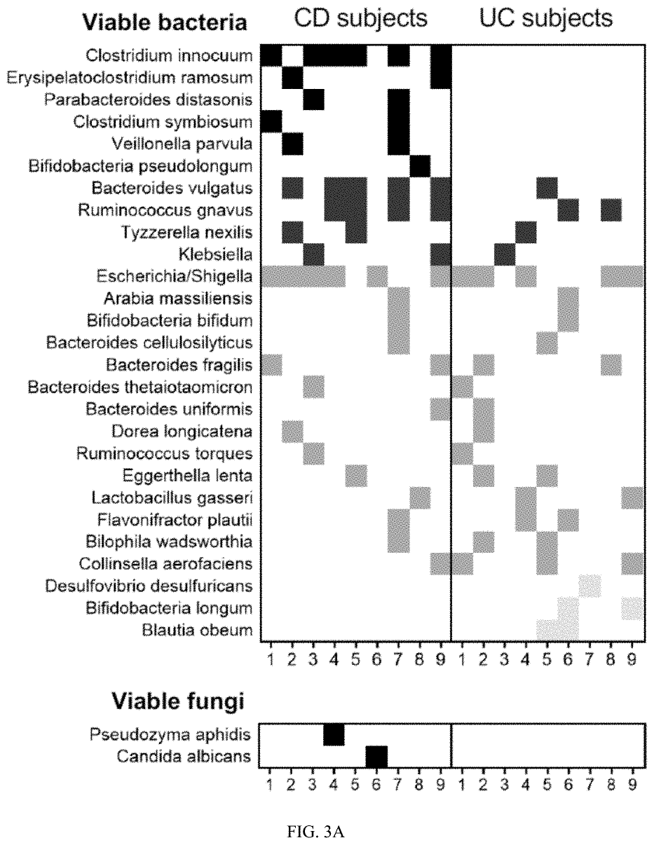

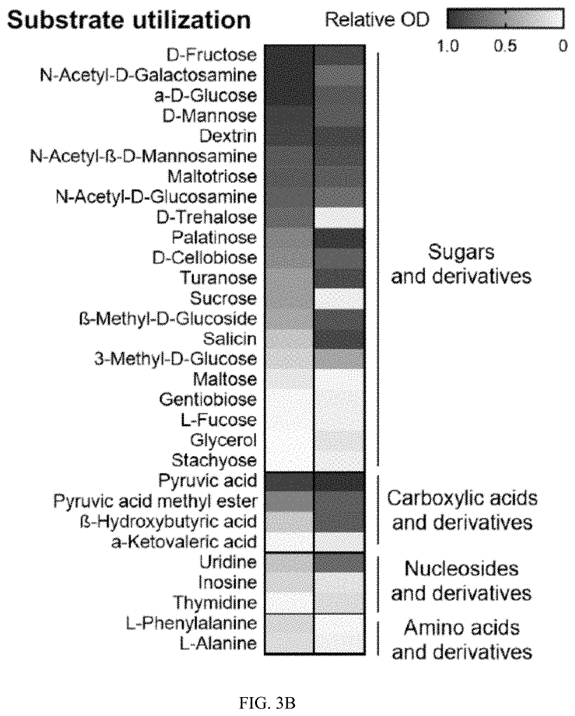

When cultivable bacterial species from MAT were stratified by disease, a subset was found exclusive to CD patients (FIG. 3A), including Clostridium innocuum, Erysipelatoclostridium ramosum, Parabacteroides distasonis, Clostridium symbiosum, Veillonella parvula, Bifidobacteria pseudolongum. One organism in particular, Clostridium innocuum, a member of the Erysipelotrichaceae family was isolated from CrF and MAT in two-thirds of the CD patients, and none from the adipose of UC patients. C. innocuum is a gram-positive, vancomycin-resistant, spore-forming member of the commensal microbiota, and is also the second most common species to cause extra-intestinal Clostridial infection, second to Clostridium perfringens. In one of the C. innocuum-negative cases, another close relative of C. innocuum within the Erysipelotrichaceae family, Erysipelatoclostridium ramosum, was isolated. We asked whether the absence of C. innocuum in UC MAT was due to a colonization preference of the ileum rather than the colon. We therefore performed cultivations from mucosal scrapings and MAT of an additional four UC patients. C. innocuum isolates were recovered from the mucosa of these cases but not from the paired MAT even though other viable bacteria were detected. This suggests that translocation of C. innocuum to MAT is specific to small bowel inflammation seen in CD. An intriguing selectivity was observed when C. innocuum in CrF was compared to uninvolved MAT. Here, visually distinct C. innocuum isolates were observed in CrF vs. uninvolved MAT in CD patients, when grown on pre-reduced chocolate blood agar. The distinguishing characteristics of C. innocuum which colonizes CrF and uninvolved MAT suggested strain variation that may either stratify these tissues, or stratify patients. Further functional characterization showed that while these isolates can utilize the same set of carbon sources including sugars, carboxylic acids, amino acids, nucleosides and their respective derivatives, each C. innocuum variant possessed different metabolic capacities (FIG. 3B). Notably, their preferences for sugar utilization were distinct, and the isolates cultivated from CrF could better metabolize amino acids. Despite the variability across the C. innocuum strains, one substrate all the isolates appeared to be able to highly use is beta hydroxybutyrate which is a breakdown product of lipid metabolism, indicating this organism is lipophilic.

To that end, we performed whole-genome sequencing (WGS) on each CD adipose isolate that appeared to have a different morphology from all the others, (each Clostridium innocuum isolate recovered from an ileal fibrosis Crohn's disease patient, a total of 6 patients wherein 3 of them each have two isolates identified), and ended up with nine unique isolates from the six C. innocuum-positive patients. The nine isolates were compared to a reference strain C. innocuum 2959, in terms of amino acid similarity percentage to the reference genome from 100% to 10% (in bidirectional best hit and/or unidirectional best hit). The assembled genomes of these isolates revealed that each isolate(s) represented a genetically different strain in each patient, and these are somewhat different from the reference strain. However, the conserved genes across all CD adipose strains fall under pathways that would suggest a competitive advantage for translocation to more toxic lipid and oxygen-rich environments such as adipose. Among these are genes for protecting against oxidative damage (superoxide dismutase/reductase), genes regulating adhesion an immune evasion (ethanolamine permease and tryptophan synthase), lipid utilization genes (monoglyceride lipase and lysophospholipase) and motility (twitching motility), which is typical of bacteria that migrate across viscous surfaces and unusual for gram-positive bacteria (Table 4).

TABLE 4
Common features of CD-derived Clostridium innocuum isolates:
the conserved genes across all isolates, with features
that confer ability to translocate (motility) and survive
in mesenteric fat (fatty acid metabolism).

| Category | Genes |
|---|---|
| Oxidative Stress | Peroxiredoxin/thiol peroxidase |
| | NADH oxidase |
| | Cystathionine beta-lyases/cystathionine |
| | gamma-synthases |
| | Manganese catalase/spore coat |
| | protein CotJC |
| | Superoxide dismutase |
| | Superoxide reductase |
| | Alkyl hydroperoxide reductase/anaerobic |
| | sulfite reductase |
| Adhesion | Peptidase M23/37 |
| | Fibronectin/fibrinogen binding protein |
| | Oligopeptide ABC transporter, substrate- |
| | binding protein OppA |
| | Oligopeptide ABC transporter, ATP-binding |
| | protein OppD |
| | Oligopeptide ABC transporter, ATP-binding |
| | protein OppF |
| | Type IV fimbrial assembly |
| Iron Acquisition | ABC-type Fe3+ transport system |
| | Iron ABC transporter permease |
| Lipid metabolism | Monoglyceride lipase |
| Toxin | Hemolysin III |
| Motility | Twitching motility |
| Metabolites | Acyl-CoA: acetate CoA-transferase |
| | Acetate kinase |
| | Tryptophan synthase |

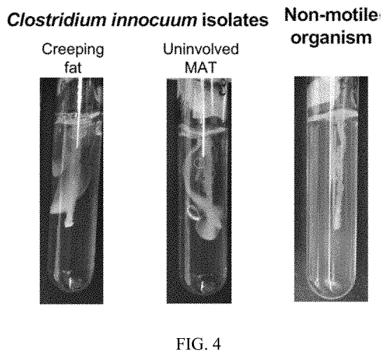

This latter feature was important to us as motility is an important characteristic to discern active versus passive migration to other sites. C. innocuum is described as a non-motile bacterium in the literature, and yet our WGS data and the reference genome of C. innocuum for the Human Microbiome Project (GenBank assembly accession: GCA_000371425.1) suggested otherwise. We tested the motility using an agar-based motility assay, and indeed, all forms of CD-derived C. innocuum demonstrated twitching motility in vitro (FIG. 4) confirming the twitching motility found in the WGS data. The C. innocuum strains also possessed the gene for type IV fimbrae assembly which is responsible for formation of type IV pilli. Previous studies have shown that the type IV pilli required for twitching motility is necessary for some pathogens to migrate, adhere to and invade epithelial cells and promote intracellular division. Twitching motility, therefore, may allow C. innocuum to translocate and migrate through a compromised epithelial barrier.

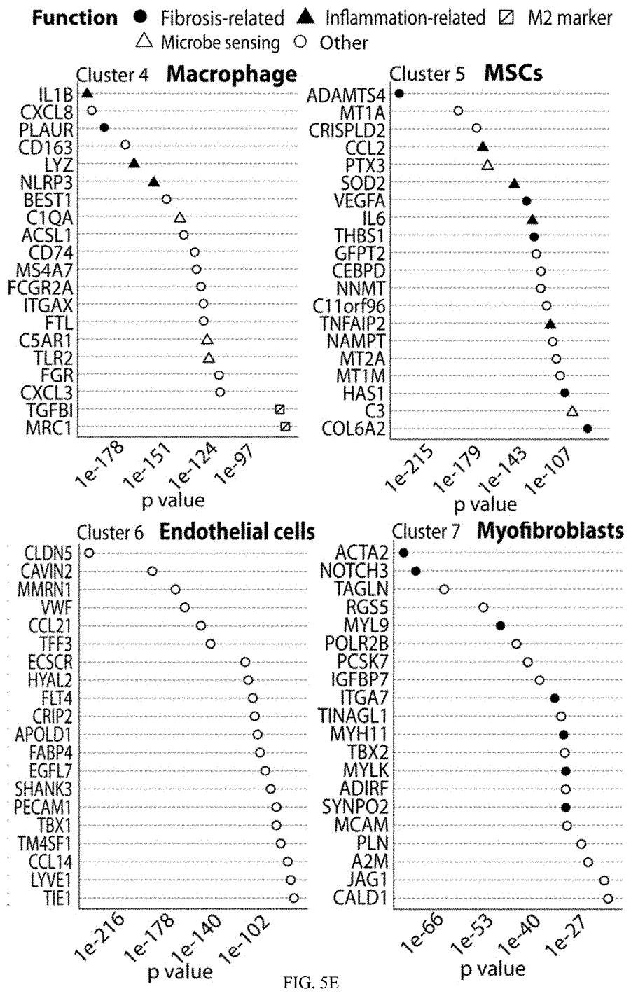

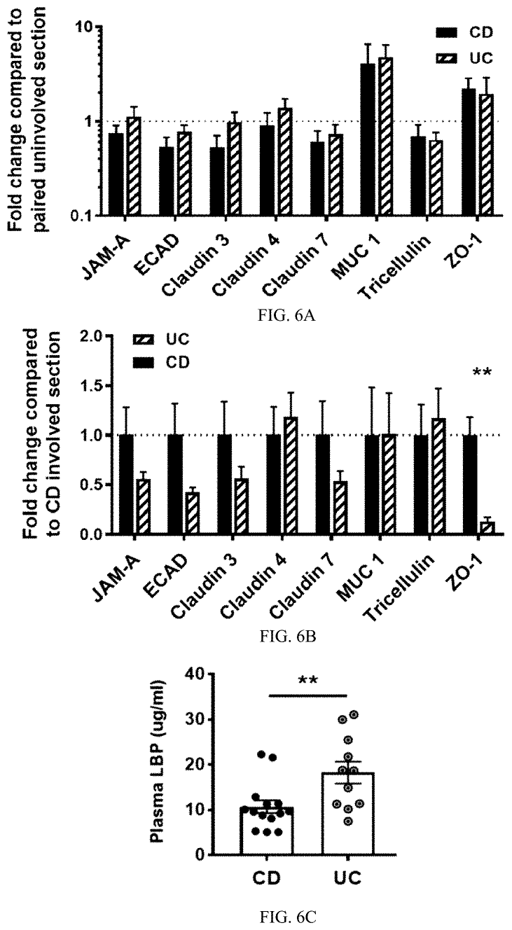

We speculated whether variations in cultivable bacteria could be attributed to differences in intestinal permeability between CD and UC. Intestinal tight junction gene expression was assessed for junctional adhesion molecule-A, E-cadherin, claudins 3, 4 and 7, Muc1, tricellulin, and zonulin-1 (ZO-1) from full thickness tissue we collected from involved and uninvolved specimens within the ileum for CD and colon for UC in each patient. (FIG. 6A). This data shows that involved ileum in CD had lower expression of all measured barrier genes compared to paired uninvolved ileum, except for Muc 1 and ZO-1. Similar trends were detected in UC between the involved and uninvolved specimens. When CD and UC involved sections were directly compared, UC trended toward decreased barrier function compared to CD, with significantly less expression of ZO-1 (FIG. 6B). The majority of these patients were medically refractory with uncontrolled inflammation, whereas the CD patients were primarily experiencing strictures which may present with less severe inflammation than medically refractory inflammatory cases. In line with this, measurements of plasma lipopolysaccharide (LPS)-binding protein (LBP), a surrogate marker of intestinal permeability that is proportionately related to the amount of circulating LPS in the blood, were significantly lower in CD patients compared to UC (FIG. 6C). Interestingly, this suggests that while there are permeability defects at the level of the gut in both CD and UC, the bacteria and their antigens are not disseminating as systemically as they do in UC. Interestingly, the histological characterization of CrF reveals extensive fibrosis throughout the adipose, yet this is not evident in UC MAT. Fibrosis, functionally, is a wound-healing response, which in the case of CrF, may be a reaction to perceived intestinal damage triggered by the presence of microbial antigen. Hence, the tissue expansion to contain further dissemination of microbes. This does not occur in UC, which may explain the significantly higher levels of LBP. Overall, these data demonstrate that translocation of viable intestinal organisms to neighboring tissues is mediated by disruption of intestinal barrier function at sites of inflammation, and further suggest that translocation of C. innocuum does not promote systemic inflammation in CD, but rather, may be a central trigger for the adipose expansion observed in CrF.

### Creeping Fat is Distinguished by Markers of Tissue Remodeling

Disease-specific and CrF-specific microbial translocation led us to believe that CrF is mediated by a highly specific host-microbe response. To determine host responsiveness in the presence of live, translocated bacteria in the mesenteric adipose, we characterized the cellular composition and transcriptional activity of stromal vascular cells (SVCs) by sequencing scRNA-seq from CrF and uninvolved MAT (u-MAT) from three CD patients and two UC patients. An average of 1,200 filtered cells per specimen were analyzed at a read depth of 175,000 reads/cell, with ˜200 unique genes per cell.

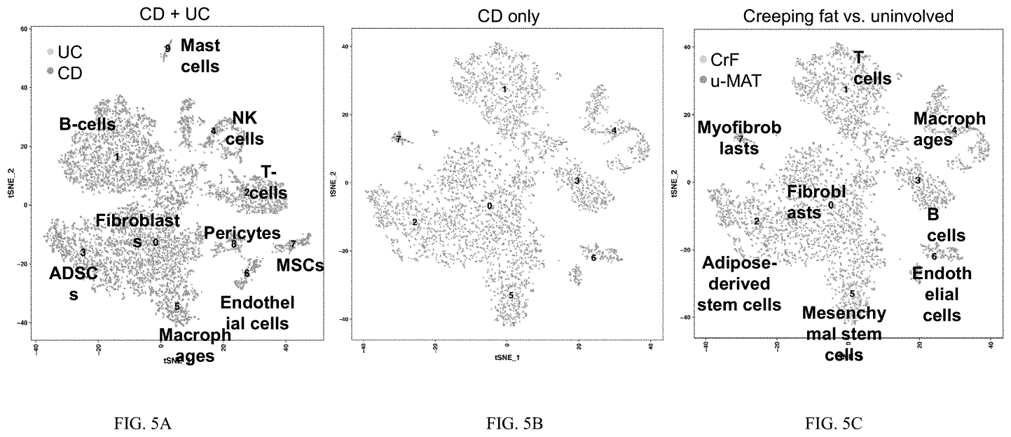

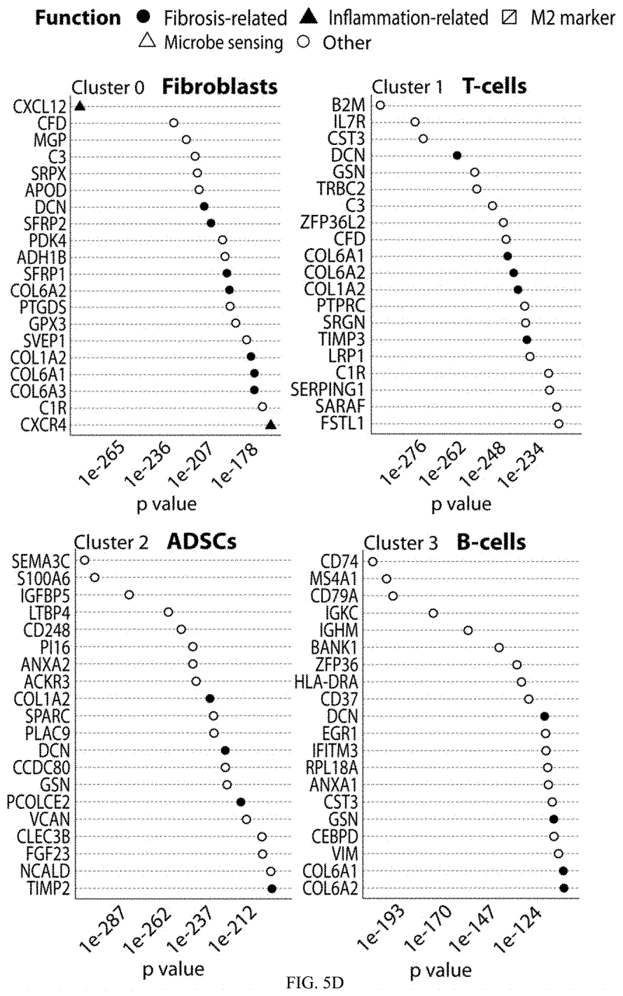

We first wanted to assess disease-specific adipose gene expression patterns comparing CD and UC, followed by a deeper analysis of the CD population alone looking at inter-individual variability, and within-disease differences of CrF vs. u-MAT. When CD and UC patients were combined for analysis, the single-cell profiles partitioned into 10 distinct cell populations as follows: cluster 0-fibroblasts, 1-B cells, 2-T cells, 3-adipose-derived stem cells (ADSCs), 4-NK cells, 5-macrophages, 6-endothelial cells, 7-mesenchymal stem cells (MSCs), 8-pericytes and, 9-mast cells in the order of most abundant to least abundant cell type. Many of these cell populations, such as macrophages, are well-characterized in adipose tissue, while others such as B cells are poorly understood. When the combined samples are pseudocolored by disease subtypes, it was clear that while CD vs. UC MAT have some overlapping clusters, there are also clusters that distinguish CD from UC adipose. The adipose milieu of CD MAT, for example, is overrepresented by structural and proliferating cells such as fibroblasts and stem cells (clusters 0 and 3), while UC is skewed toward immune cells such as B cells, and mast cells (clusters 1 and 9). Notably, the gene expression profiles show concomitant differences further indicating subpopulations unique to disease. In this case, one of the most pronounced distinctions was within the macrophage cluster between CD and UC. CD macrophages, while highly expressing pro-inflammatory IL-1β and lysozyme, these were only two of three pro-inflammatory genes in the top 20 expressed genes. The remaining genes were either related to microbe-sensing, normal macrophage function, or M2 markers such as TGF-β and mannose receptor 1 (MRC1). This suggests CD adipose macrophages are not of the canonical M1 phenotype and may be more closely related to M2-polarized macrophages. UC specimens, also highly expressed IL-1β and lysozyme, however, 10 of the top 20 genes were pro-inflammatory and devoid of M2 markers. This is consistent with known cellular patterns observed in intestinal tissue, where stricturing disease often undergoes a transition from overt inflammation to an M2 macrophage-mediated wound-healing response that, when uncontrolled, leads to fibrosis. This finding lends support to our notion that the fibrotic wound-healing milieu in stricturing CD is not restricted to the intestines and may be a physiological response to certain bacteria. FIGS. 5D and 5E show the top 20 genes expressed in the panel in FIG. 5C, which shows the high expression of extracellular matrix gene expression across almost all clusters (pro-fibrosis genes), and the high expression of microbe-sensing genes (showing these bacteria can persist in adipose and affect host cellular phenotypes).

We subsequently sought to understand unique features of the CD adipose specimens that may guide us toward a better understanding of CrF. CD-derived adipose SVC's revealed eight distinct cell types: 0-fibroblasts, 1-T cells, 2-ADSCs, 3-B cells, 4-macrophages, 5-MSCs, 6-endothelial cells, 7-myofibroblasts. The dominant antigen-presenting cell in u-MAT and CrF were macrophages, which, among the top 20 highly expressed genes, were upregulated for anti-microbial or microbial sensing genes such as lysozyme, complement factors C1 QA and C5AR1, and toll-like receptor 2. These expression profiles are indicative of chronic exposure to microbial antigen rather than transient exposure at time of surgery (i.e. contamination). These data reinforce that the live microorganisms detected are not contaminants. Furthermore, the MAT of CD patients is transcriptionally upregulated for collagen production in five of the eight cell types detected and this was not seen in UC MAT. Interestingly, but not unexpectedly, inter-individual differences in cellular distribution across clusters was observed between patient specimens. However, each cell type was represented in each patient.

Ultimately, within the CD MAT specimens, we wanted to determine whether unique cellular signatures existed between CrF and u-MAT. Visualizing the data in this manner clearly delineated cell clusters unique to CrF. While all cell types were represented in both CrF and u-MAT, CrF was clearly skewed toward fibroblasts, mesenchymal stem cells, and myofibroblasts. This is notable given that these cells are also dominant in intestinal fibrosis, further suggesting cross-talk between the fibrotic wound-healing response of CrF and the ileum. u-MAT, on the other hand, was skewed toward adipose-derived stem cells (ADSCs). T cells, B cells, macrophages, and endothelial cells were represented between CrF and MAT, however differential gene expression profiles were quite different. Within these cell types, CrF was significantly upregulated for extracellular matrix pathway genes compared to u-Mat, as well as for anti-microbial sensing pathways (Table 5).

TABLE 5
Summary of upregulated genes in different cells within the CD MAT specimens.

| | | Anti-microbial | Inflammatory | Extracellular | Wnt |
|---|---|---|---|---|---|
| Cluster # | Cell types | sensing | pathways | matrix | signaling |
| 0 | Fibroblasts | | | ↑↑↑ | ↑ |
| 1 | T-cells | ↑ | ↔ | ↑↑↑ | |
| 2 | Adipose-derived | | | ↑↑ | ↓ |
| | stem cells | | | | |
| 3 | B-cells | ↑↑ | | ↑↑ | |
| 4 | Macrophages | ↑↑↑ | ↑↓ | ↑ | |
| 5 | Mesenchymal | ↑ | | ↑↑ | ↑ |
| | stem cells | | | | |
| 6 | Endothelial cells | | | | |
| 7 | Myofibroblasts | | | ↑↑↑ | ↑ |

↑ Upregulation;
↓ Downregulation;
↑ p < 0.005;
↑↑ p < 10⁻⁶;
↑↑↑ p < 10⁻¹⁰

Within the proliferative cell compartment unique to CrF, pathways related to Wnt signaling were also upregulated compared to u-MAT. Taken together these results suggest CrF is distinguished from u-MAT by increased sensitivity to microbiota or microbial products, plausibly due to increased translocation and sequestration to this compartment. CrF is concomitantly characterized by M2 macrophage differentiation and a strong upregulation of ECM deposition and cellular proliferation, likely explaining both the rigidity and hyperplasticity of the tissue. The absence of this cellular profile in UC, despite increased permeability, suggests the required signal is not present in the colonic MAT to trigger CrF. This signal may be a specific microorganism, such as C. innocuum.

### CrF Bacteria Promote Pro-Fibrotic Phenotypes in Primary Macrophages and Fibroblasts

The question remains how translocated micro-organisms persist in CrF without stimulating continuous overt inflammation. They may have evolved ways to evade the immune system, or they may directly mediate the M2 macrophage response. To further delineate the role of translocated bacteria in CrF pathophysiology, and specifically of C. innocuum, we examined whether the presence of candidate bacteria can stimulate these pathways in vitro using primary human macrophages, stem cells and fibroblasts. While CrF-associated C. innocuum was our leading candidate for mechanistic exploration, we also considered whether a consortium of selected CD-exclusive cultivable organisms (C. innocuum, E. ramosum, Parabacteroides distasonis and Bifidobacterium pseudolongum) would have a synergistic effect on the identified host cell populations.

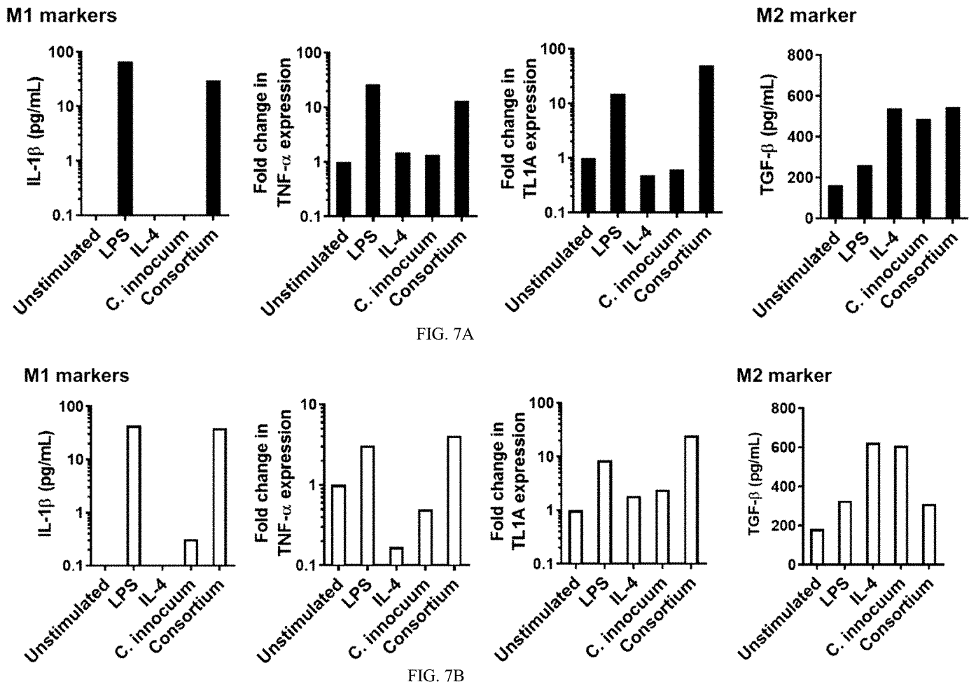

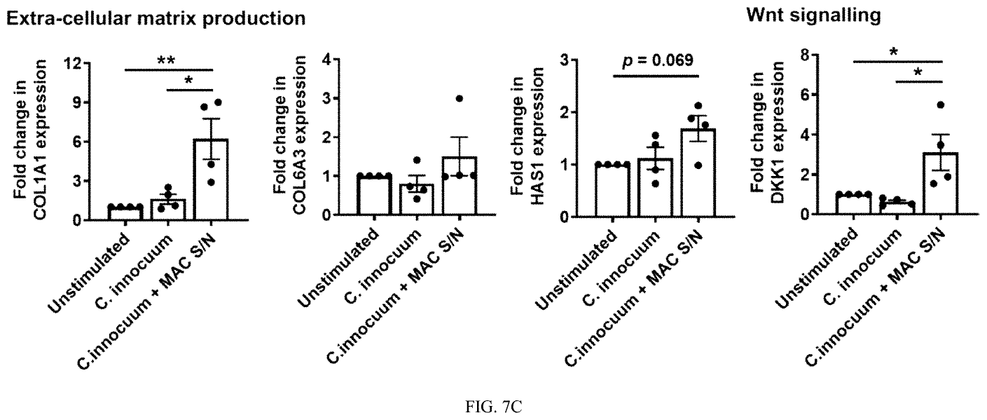

The influence of these CD-associated isolates on macrophage phenotype was investigated using peripheral blood monocytes (PBMCs) from CD patients and healthy volunteers following the approach of Schirmer et al. of 2016. The PBMCs were differentiated into macrophages and exposed to known stimuli of M1 and M2 responses (LPS and IL-4, respectively), or bacterial lysates from freshly grown cultures. Macrophages are known to exhibit morphology changes when differentiated to M1 or M2-like subtypes: with M1's displaying a typical round, spiked morphology, and M2's forming elongated spindles. Indeed, when we exposed macrophages to LPS, they maintained a rounded morphology, while exposure to IL-4 resulted in the elongated morphology over 24 hrs. When the cells were then exposed to C. innocuum alone, they exhibited even more pronounced elongated morphology than with IL-4 exposure, while macrophages exposed to C. innocuum as part of a CD-associated consortium consisted of both M1 and M2 morphologies. To confirm the macrophage polarization state, secreted markers of wound-healing (TGF-β) and pro-inflammatory (IL-1β, TNF-α and TL1A) phenotypes were quantified by ELISA and RT-qPCR. Here, IL-4 and C. innocuum exposure induced higher levels of TGF-β than LPS-stimulation, but failed to stimulate IL-1β, TNF-α or TL1A (FIG. 7A). Whereas lysates from the CD consortium induced all three cytokines. This same trend was observed in PBMC-derived macrophages isolated from healthy volunteers (FIG. 7B). In summary, these data support that C. innocuum polarizes an M2 macrophage phenotype that is consistent with the scRNA-seq profile of CrF macrophages.

We next sought to determine the effects of these polarized macrophages on the end phenotype of CrF, which is ultimately cellular proliferation and tissue fibrosis. We hypothesized that the expansion of CrF proceeds through a signaling cascade initiated by C. innocuum stimulated M2 macrophages. These macrophages would then signal to proliferative cells in the fibroblast or stem cell compartment to expand, driving a wound-healing response manifesting as hyperproliferation and fibrosis. Fibroblasts and stem cells comprised roughly half the cells in the SVC fraction of CrF as determined by our scRNA-seq data, therefore, we isolated these cells from resections for primary cell culture and exposed them to C. innocuum stimulated macrophage supernatant, or C. innocuum lysate directly. Our readouts were based on the most differentially expressed CrF genes in this cell population, which were related to the formation of extra-cellular matrix (ECM) and the Wnt signaling pathway specifically collagen 1A (Col1A1), hyaluranon synthase 1 (HAS1), and Dickkopf 1 (Dkk1). Therefore, we measured expression of these genes upon direct or indirect C. innocuum stimulation by RT-qPCR.

C. innocuum lysates alone were insufficient in modulating any of these genes, however the M2 conditioned media significantly increased expression of all three genes compared to unstimulated and direct C. innocuum exposure (FIG. 7C). ScRNA-seq showed that DKK1, which attenuates proliferation by inhibiting the canonical Wnt pathway, was upregulated in ADSC's and downregulated in fibroblasts. However, in vitro, we found significant upregulation of DKK1 with exposure to the conditioned media. This may be reflective of the relative proportion of ADSCs to fibroblasts in our culture, or may reflect true biological phenomenon where proliferation is often balanced by anti-proliferative signals.

Together, however, these in vitro data demonstrate the potential to re-create important aspects of the CrF phenotype identified by scRNA-seq through cultivation of primary cells exposed to specific patient-derived bacterial stimuli. Here we identify for the first time, C. innocuum as a hallmark signature of CrF that is both cultivable and identifiable by sequencing in both the ileal mucosa and mesenteric adipose of CD patients, and can stimulate M2 macrophage polarization in this adipose depot. M2 macrophages have long been implicated in the wound-healing and fibrotic responses characterized by increased ECM deposition in tissues. Indeed, we find that C. innocuum stimulated M2 macrophages drive upregulation of ECM genes in fibroblasts and ADSCs, providing a bacterial mechanism that offers new insight into this mysterious complication of Crohn's disease.

While the context of this study is a unique extraintestinal phenomenon in CD, the findings herein lend new insights into the roles of adipose tissue in the human body and how the gut microbiome may influence its behavior. The primary reputation of adipose as a storage form for excess calories, while necessary, suggests a passive role in the body. However, we show here, that this natural plasticity of adipose may actually have another, equally important purpose, which is to protect the body from dissemination of harmful antigens at sites of inflammation or injury. Our findings show that the chronic inflammation present in CD leads to impaired barrier function forming a permissive interface for bacterial translocation. While many bacteria translocate, not all survive in non-native environments such as adipose. Here, we identify for the first time, a potential microbial mechanism explaining the hyperplastic expansion of so-called creeping fat, driven by a gut-derived bacterium that translocates and survives in mesenteric adipose, Clostridium innocuum. Its ability to translocate was unique to CD patients, and presents as different strains in creeping fat compared to adjacent uninvolved adipose. Functionally, C. innocuum appears to promote M2 macrophage polarization, which, is well-established in the literature to promote a fibrotic wound-healing response through expression of TGF-β. Indeed, we demonstrate enhanced TGF-β expression in C. innocuum stimulated macrophages and upregulated gene expression of extracellular matrix genes when patient-derived proliferative cells were exposed to the macrophage conditioned media. These data present the first mechanism whereby C. innocuum may trigger and sustain the development of creeping fat via signaling through M2 macrophages.

We confirm in situ the prominence of M2 macrophages and pro-fibrotic fibroblasts and stem cells through single-cell RNA sequencing of creeping fat and paired adjacent uninvolved mesenteric fat in CD patients, as well as paired involved and uninvolved mesenteric fat from UC patients as comparisons of how the mesenteric adipose cellular milieu differs in the two prominent forms of IBD. To our knowledge, this is the first ever single-cell RNA sequencing dataset in these tissues. This dataset further identifies the major cell types present and their gene expression profiles which lent additional insights. Among these are the robust expression of anti-microbial genes in all of the immune cells identified, particularly macrophages and B cells. While we were confident in our rigorous sample collection protocol and aseptic technique, these data confirmed that the exposure of the mesenteric adipose tissue to live bacteria was established, rather than transient contamination. To our surprise, we also found that while fibroblasts and stem cells most strongly expressed genes related to extracellular matrix genes, every cell type, even the immune cells, had upregulated collagen gene expression, and warrants further investigation.

In this study, we cannot exclude the role of the other translocated microbiota in influencing the creeping fat phenotype, of which there were several. For example, Ruminococcus gnavus, was another CD-dominant signature we identified, however it was excluded from our investigations because it was also found in two UC patients. However, we donated a representative CD-specific R. gnavus isolate for use in the recent study on intestinal fibrosis by Jacob et al., which found it could strongly promote fibroblast proliferation in an in vitro wound-healing assay. R. gnavus has also been reported in several recent studies to bloom in CD patients experiencing inflammatory flares. We also cannot exclude the role of fungi. ITS sequencing clearly showed an abundance of fungal sequences in the mesenteric adipose, and an interesting CD-specific signature. However, we were unable to isolate these fungi in culture. Whether this reflects that fungi cannot survive in the adipose tissue, or that our fungal cultivation methodologies, despite recapitulating established protocols, failed to capture the viable community remains to be determined, and is an area of active pursuit in our lab. Finally, from a host perspective, we must appreciate that we discovered a rich milieu of immune cells in creeping fat in response to translocated bacteria. While we focused primarily on macrophages due to their distinguishing M1/M2 phenotype between CD and UC mesenteric adipose, T-cells numerically represented a larger population than the macrophages. This was surprising to us given the expansive literature on adipose tissue macrophages, and relative dearth of information about the role of adaptive immune cells in adipose tissue and specifically creeping fat. Similarly, B-cells were a clear cell cluster, and their gene expression pattern reflected B-cell maturation which is often a response to bacterial stimulation. While characterization of the functional role of each immune cell population and their reactivity to each of the bacterial candidates in creeping fat is beyond the scope of this current study, the data presented here form a strong foundation for, and warrants, pursuing the origin and role of these immune cells in extraintestinal sites in CD. Despite these further questions, the data presented here helps answer the long-standing question about whether creeping fat in CD is harmful or beneficial. It is both. What begins as a reaction to intestinal injury and bacterial dissemination, aiding the body's protective response and limiting the collateral damage of systemic antigen exposure, appears to have no off-switch in the presence of continual microbial exposure, and specifically C. innocuum. This wound-healing response, in turn, leads to significantly fibrotic mesenteric adipose encasing the underlying ileum which, at the time of resection, is also significantly fibrotic. Therefore, strategies to therapeutically target or outcompete the intestinal reservoir of C. innocuum in high-risk patients, may offer an avenue for preventing or attenuating the fibrotic cascade.

### Procedures

### Human Subjects

Surgical resection specimens were collected from patients with a diagnosis of Crohn' s disease or ulcerative colitis undergoing intestinal resection. Patients provided informed consent during their pre-operative visit, and the study was approved by the Cedars-Sinai Medical Center Institutional Review Board. Exclusion criteria included patients under 18, unwilling to provide informed consent, antibiotic or anti-fungal use in the six weeks prior to surgery, or individuals with colorectal cancer or undergoing chemoradiation therapy. Clinical characteristics and demographics of the patient cohort are detailed in Table 2.

### Tissue Collection

Whole blood, resected intestinal tissue, and attached mesenteric adipose were aseptically collected from CD and UC patients undergoing bowel resection. Specimens were transferred directly from the operating room to the biosafety cabinet within 20 minutes of resection and transported in sterile containers. The ends of the resected bowel specimen were immediately sutured closed in the operating room to prevent any contamination from luminal contents onto the mesenteric adipose. Upon arrival to the biosafety cabinet, the specimen was rinsed twice in sterile PBS. Intestinal and adipose tissues were subsampled for bacterial community profiling by 16S rRNA sequencing, fungal community profiling by ITS sequencing and microbial cultivation. 0.5 g MAT was trimmed from various regions of the mesenteric adipose depot to capture any regional variability of translocation for each of the microbial experiments. Remaining MAT was reserved for stromal vascular cell isolation and for RNA isolation. The lumen of the intestine was rinsed with sterile PBS. 0.1 g intestinal tissue was stored in Trizol (Invitrogen, USA) for RT-qPCR, and mucosal scrapings were collected for microbial characterization. Specimens designated for sequencing, RT-qPCR, or excess samples were stored at −80° C. until use, others were processed within 2 hr of surgery. Both adipose and intestinal samples were submitted for histology.

### Microbial Cultivation and Identification

MAT and mucosal scrapings were homogenized by standing mortar and pestle (Fisher Scientific, USA) in sterile PBS using aseptic techniques. Samples were serially diluted and plated on the following media with 1.2% agar in both aerobic and anaerobic conditions: chocolate blood (CBA; Becton Dickson, USA), Lactobacilli MRS (Becton Dickson, USA), brain heart infusion media (BHI; Sigma, USA), Brucella (BRU; Hardy Diagnostics, USA) with 0.5% pyruvate, 0.5% taurine and 0.05% ammonium iron citrate, reinforced Clostridial media (RCM; Becton Dickson, USA), Bacteroides bile esculin (BBE; Anaerobe Systems, USA) and Sabouraud dextrose (SAB; Hardy Diagnostics; USA) with and without the addition of olive oil post-inoculation. BHI, BRU and RCM were supplemented with 5 mg/L hemin and 0.5 mg/L vitamin K. All the plates were incubated at 37° C. except for SAB, which was cultured at room temperature. Distinct colony forming units (CFUs) were re-steaked at day 4 and 7 post-incubation. Colony PCR was performed with full length 16S or ITS primers (Table 6). Amplification was carried out using the iTaq DNA polymerase kit (Bio-Rad, USA). Amplicons were submitted to Laragen for Sanger sequencing. Sequence traces were examined in FinchTV v1.4, and the resultant trimmed reads were identified by Microbial BLAST.

TABLE 6
List of primers for bacterial and
fungal characterization.

| Target | Primers | Sequence (5′-3′) |
|---|---|---|
| Bacterial 16S rDNA | 27F | AGAGTTTGATCMTGGCTCAG |
| (full length) | | (SEQ ID NO: 1) |
| | 1492R | TACGGYTACCTTGTTACGACTT |
| | | (SEQ ID NO: 2) |
| Bacterial 16S rDNA | 515F | GTGYCAGCMGCCGCGGTAA |
| (V4 region) | | (SEQ ID NO: 3) |
| | 806R | GGACTACNVGGGTWTCTAAT |
| | | (SEQ ID NO: 4) |
| Fungal ITS | ITS1 | TCCGTAGGTGAACCTGCGG |
| (full length) | | (SEQ ID NO: 5) |
| | ITS4 | TCCTCCGCTTATTGATATGC |
| | | (SEQ ID NO: 6) |
| Fungal ITS | ITS1F | CTTGGTCATTTAGAGGAAGTAA |
| (ITS1 region) | | (SEQ ID NO: 7) |
| | ITS2 | GCTGCGTTCTTCATCGATGC |
| | | (SEQ ID NO: 8) |

### Metabolic Properties of Bacterial Isolates

Bacteria resuspended in inoculating fluid (Biolog, USA) were added to AN MicroPlate (Biolog, USA) with 95 distinct carbon sources as per manufacturer's instruction. Plates were incubated in GasPak EZ anaerobic pouch system (BD, USA) at 37° C. Growth was measured colorimetrically by microplate reader (BMG LABTECH, Germany) after 48 ah incubation.

### DNA Extraction for Microbiota and Mycobiota Profiling

DNA was extracted from mucosal scrapings and adipose tissue using the DNeasy PowerSoil Kit (QIAGEN, USA) with additional steps to maximize cell lysis. Samples for microbiota profiling were added to lysis tubes with 400 μg proteinase K and homogenized at 5 m/s for 2 min. This was followed by heat treatment at 95° C. for 15 min and centrifugation at 16,000×g for 5 min at 4° C. Supernatant was transferred to a new tube and reserved for later use. 300 μL fresh lysis buffer was added back to the lysis tube for a second round of bead beating and heating. Supernatant from both rounds of cell lysis were pooled for DNA isolation as per manufacturer's protocol. Tissue aliquots reserved for mycobiota profiling were first homogenized at 6 m/s for 1 min in tubes containing 2.8 mm ceramic beads (Omni, USA), 50 mM Tris buffer (pH 7.5), 1 mM EDTA and 0.2% β-mercaptoethanol. 1000 U/ml lyticase was added to the mixture and incubated at 37° C. for 30 min with gentle agitation every 5 min, followed by centrifugation at 16,000×g for 5 min at 4° C. Cell pellet was processed in the same manner as samples for bacterial profiling. DNA extracts were then submitted to the HighThroughput Sequencing and Genotyping Unit at the University of Illinois at Urbana-Champaign for bacterial sequencing and the Genomics Core at Cedars-Sinai Medical Center for ITS sequencing respectively.

### 16S rRNA and ITS Sequence Analysis

R packages were used to process and analyze 16S and ITS sequences. Paired-end reads were quality filtered, trimmed, merged, denoised, chimera filtered, and binned into sequence variants using DADA2 v1.5.8. Average number of 16S and ITS reads per sample was 5,760 and 2,369, respectively. Samples with less than 1,000 reads were removed from analysis. 16S sequence variants were aligned to the Greengenes reference database v13.8 and taxonomically assigned with a minimum bootstrap confidence level of 80. ITS sequence variants were classified using the Targeted Host Fungi ITS1 database v1.6. Sequence variants unresolved for taxonomic classification and singletons were omitted from further analyses. Samples were rarefied to the minimum read count to account for uneven sampling effort. Phyloseq v1.22.3 was used to assess α and β diversity measures. Bray-Curtis distance between samples were visualized by principal coordinate analysis.

### Whole Genome Sequencing and Analysis

Clostridium innocuum isolates were grown on pre-reduced chocolate blood agar at 37° C. for 36 hours. Colonies were scraped from the plate with a sterile 10 μl inoculating loop and the genomic DNA was extracted using the DNeasy PowerSoil Kit (QIAGEN, USA). Purified DNA was sent to the Microbial Genome Sequencing Center at University of Pittsburgh for library preparation, followed by whole genome sequencing on the Illumina NextSeq 550 flow cell. Sequences were assembled and annotated using the Pathosystems Resource Integration Center (PATRIC) software version 3.5.43. Comparison between annotated genes across C. innocuum genomes was completed by an implementation of RAST's Sequence-based Comparison tool within PATRIC. This tool analyzed each gene based on protein similarity using BLASTP and marked each gene as either unique, a unidirectional best hit or a bidirectional best hit when compared to the reference genome C. innocuum 2959 (a reference genome for the Human Microbiome Project).

### Lipopolysaccharide-Binding Protein (LBP) Assay

Whole blood collected at the time of surgery and separated for plasma by centrifugation at 1200×g for 10 min at 4° C. Plasma LBP was measured by ELISA (Hycult Biotech, The Netherlands) as per manufacturer's instructions. LBP was measured colorimetrically by microplate reader (BMG LABTECH, Germany).

### Stromal Vascular Cell (SVC) Isolation from Adipose Tissue for Single-Cell RNA Sequencing (ScRNA-seq)

Adipose tissue was minced into 3 mm pieces and subjected to three 20 min rounds of collagenase digestion for 45 min at 37° C. with continuous rotation. Collagenase buffer consisted of 1× PBS containing calcium and magnesium, 2% bovine serum albumin (BSA; MP Bio, USA), 0.2 mg/mL DNase I (Sigma Aldrich, USA), and 1 mg/mL collagenase II (Invitrogen, USA). Following digestion, cells were incubated with 0.01 M EDTA for an additional 10 min then filtered through a 100 μM cell strainer (Fisherbrand, USA) and centrifuged at 1500 rpm for 5 min. The pellet was resuspended in 1× RBC Lysis Buffer (Invitrogen, USA) as per manufacturer's instruction. The samples were centrifuged as above, and the final pellet was converted to a single-cell suspension and frozen for later analysis such that all samples could be run at the same time. The cell-freezing protocol for scRNAseq was developed in conjunction with Bio-Rad Genomics. Briefly, the cell pellet was thoroughly mixed by pipetting up and down 10 times. Cells were counted a total of 4 times for each cell preparation to ensure accuracy of total cell count and viability. Aliquots of 5*10\hat{ }6 cells/ml were prepared in chilled cryopreservation medium (DMEM+20% FBS+10% DMSO) and placed in a 4 C pre-chilled CoolCell FTS30 and placed in a −80 C freezer for at least 4 hrs. After 4 hrs the cryovials were transferred to liquid nitrogen for long-term storage.

### Single-Cell RNA Sequencing

Dissociated SVCS were first diluted to 2500 cells/uL in PBS with 0.1% BSA. Cells were individually partitioned and co-encapsulated with barcodes into subnanoliter oil droplets using the ddSEQ single-cell isolator (Bio-Rad, USA) as per manufacturer's instructions in the Illumina Bio-Rad SureCell WTA 3′ Library Prep Kit. Following cell isolation, the droplets were transferred to a 96-well PCR plate for cell lysis, barcoding, reverse transcription using a thermal cycler. The droplet emulsion was then disrupted for generation of second strand cDNA, followed by fragmentation, tagging and amplification of cDNA. Single-cell-barcoded cDNA libraries were sequenced on the Illumina NextSeq platform at an average read depth of 175,000 reads/sample.

### Single-Cell RNA Sequencing Analysis

Cell counts were generated with the bcbio Python toolkit. Reads were assigned per cell via the cellular barcodes, and per gene via the UMIs, using the umis toolkit. Reads were quasi-mapped to the Ensembl GRCh38 transcriptome (Release 90, August 2017) using Rapmap. Only cells containing at least 1,000 reads were analyzed. The bcbioSingleCell R package was used to perform cell quality control analysis and filtering prior to clustering. The distributions of reads per cell, UMIs per cell, genes per cell, and mitochondrial ratio per cell were used to remove low quality cells from analysis.

Clustering analysis was performed with the Seurat R package. Counts were log normalized and scaled per cell to account for variations in sequencing depth. Linear dimensional reduction was performed using PCA on the most variable genes detected; these are determined via binned z-scores based on the average expression and dispersion for each gene. Non-linear dimensional reduction was performed using t-SNE and UMAP. Differential expression analysis was performed using edgeR. All code will be made publicly available on GitHub.

### Immunogenicity Assays with Monocyte-Derived Macrophages

Macrophages were generated from peripheral blood monocytes isolated from patients and healthy controls. Healthy controls included individuals without any diagnosed GI disorders or on antibiotics in the six weeks prior to blood draw. Blood fractions were first separated by density gradient using Ficoll-Paque Premium (GE Healthcare). Monocytes-enriched buffy coat was collected, and platelets were discarded by 3 washing steps in PBS. Monocytes were seeded in RPMI with 10% FBS, 4 mM L-Glutamine, 100 I.U./ml penicillin and streptomycin in tissue culture treated plates for 7 days in the presence of 50 ng/ml M-CSF (PeproTech USA). Macrophages were incubated with 25 μg/mL lysate from bacterial isolates cultivated from MAT for 24 h. For M1 and M2 polarization, macrophages were stimulated with 50 ng/ml LPS and 25 ng/ml IL-4, respectively. Phase contrast images of macrophages were acquired on an inverted microscope (Echo Labs, USA). Supernatant was reserved for further co-culture experiments or quantification of IL-1β by ELISA (R&D Systems, USA). Macrophages were lysed and stored in Trizol (Invitrogen, USA) for RT-qPCR.

### Co-Culture Assays Involving Adipose-Derived Stem Cells (ADSCs) and Fibroblasts

Cells were prepared as described previously. ADSCs were co-cultured with 25 μg/mL bacterial lysates or supplemented with 20% macrophage supernatant (collected after bacterial stimulation) from CD patients. Cells were lysed after 36 h incubation and stored in Trizol (Invitrogen, USA) for RT-qPCR.

### Real-Time Quantitative PCR

Total RNA was extracted from intestinal tissue and cell culture homogenate in Trizol (Invitrogen, USA), and purified using the RNeasy Mini Kit (QAGEN, USA). The iScript cDNA Synthesis kit (Bio-Rad, USA) was used to reverse transcribe RNA for RT-qPCR. SYBR Green Supermix (Bio-Rad, USA) and the CFX Connect System were used to detect amplification of target genes (Table 7), using the following protocol: 95° C. for 3 minutes; 40 cycles of 95° C. for 15 seconds and 58° C. for 40 seconds. Relative expression of target genes was normalized to GAPDH, ACTB or RPL37A and quantified by 2^{ΔΔ}CT.

TABLE 7
List of primers used in characterization
of host metrics.

| Target | Primers | Sequence (5′-3′) |
|---|---|---|
| GAPDH | GAPDH-F | CTCTGCTCCTCCTGTTCGAC |
| | | (SEQ ID NO: 9) |
| | GAPDH-R | TTAAAAGCAGCCCTGGTGAC |
| | | (SEQ ID NO: 10) |
| Claudin-3 | Cldn3-F | CACGCGAGAAGAAGTACACG |
| | | (SEQ ID NO: 11) |
| | Cldn3-R | CCTGCGTCTGTCCCTTAGAC |
| | | (SEQ ID NO: 12) |
| Claudin-4 | Cldn4-F | CATCTCCTCTGTTCCGGGTA |
| | | (SEQ ID NO: 13) |
| | Cldn4-R | ATCCACTCTGCACTTCCCAG |
| | | (SEQ ID NO: 14) |
| Claudin-7 | Cldn7-F | GCAAAATGTACGACTCGGTG |
| | | (SEQ ID NO: 15) |
| | Cldn7-R | CACAAACATGGCCAGGAAG |
| | | (SEQ ID NO: 16) |
| Claudin-12 | Cldn12-F | GCTGTTTTGGAACTGTCAGG |
| | | (SEQ ID NO: 17) |
| | Cldn12-R | TTCCACACAGGAAGGAAAGG |
| | | (SEQ ID NO: 18) |
| E-cadherin | Ecad-F | GCCGAGAGCTACACGTTCAC |
| | | (SEQ ID NO: 19) |
| | Ecad-R | GTCGAGGGAAAAATAGGCTG |
| | | (SEQ ID NO: 20) |
| Junctional | JamA-F | TCATATTGGCGATCCTGTTG |
| adhesion | | (SEQ ID NO: 21) |
| molecule-A | JamA-R | AGGCACAGGACAACTTCACA |
| | | (SEQ ID NO: 22) |
| Zonula | ZO1-F | ACAGCAATGGAGGAAACAGC |
| occludens-1 | | (SEQ ID NO: 23) |
| | ZO1-R | CCCCACTCTGAAAATGAGGA |
| | | (SEQ ID NO: 24) |
| Mucin-1 | Muc1-F | CCCTCCCAGTGTGCAAATAAG |
| | | (SEQ ID NO: 25) |
| | Muc1-R | GAACGGTGTCGTCGAAACAG |
| | | (SEQ ID NO: 26) |
| Trefoil factor-1 | TFF1-F | TGCCGCCGAAAGAACTACG |
| | | (SEQ ID NO: 27) |
| | TFF1-R | TGGGGTACTCGCTCATAGGAT |
| | | (SEQ ID NO: 28) |
| Tricellulin | Tricel-F | GGCAGCTCGGAGACATAGAG |
| | | (SEQ ID NO: 29) |
| | Tricel-R | TTTGCTGTTCTCAGTTCCTTGA |
| | | (SEQ ID NO: 30) |
| RPL37A | RPL37A-F | ATTGAAATCAGCCAGCACGC |
| | | (SEQ ID NO: 31) |
| | RPL37A-R | AGGAACCACAGTGCCAGATCC |
| | | (SEQ ID NO: 32) |
| Collagen I | Col1A1-F | GTCACCCACCGACCAAGAAACC |
| | | (SEQ ID NO: 33) |
| | Col1A1-R | AAGTCCAGGCTGTCCAGGGATG |
| | | (SEQ ID NO: 34) |
| Collagen VI | COL6A3-F | AAGCCCTGACTGGTATCCCT |
| | | (SEQ ID NO: 35) |
| | COL6A3-R | CAGCCGCACCATTTTTGACA |
| | | (SEQ ID NO: 36) |
| Hyaluranon | HAS1-F | TCAGCCCAAGATTCTTCAGTC |
| synthase 1 | | (SEQ ID NO: 37) |
| | HAS1-R | GAACGAGGAGAAAGCAGGAC |
| | | (SEQ ID NO: 38) |
| Dickkopf 1 | DKK1-F | CCTTGGATGGGTATTCCAGA |
| | | (SEQ ID NO: 39) |
| | DKK1-R | CCTGAGGCACAGTCTGATGA |
| | | (SEQ ID NO: 40) |
| TNF-α | TNFα-F | ATGAGCACTGAAAGCATGATCC |
| | | (SEQ ID NO: 41) |
| | TNFα-R | GAGGGCTGATTAGAGAGAGGTC |
| | | (SEQ ID NO: 42) |
| TL1A | TL1A-F | CTTCCTTGCAGGACTCACCAC |
| | | (SEQ ID NO: 43) |
| | TL1A-R | GCTGATGTGAAGGTGCAAACTC |
| | | (SEQ ID NO: 44) |

### Quantification and Statistical Analysis

Statistical analysis was performed using GraphPad Prism software v 7.02. Data were assessed for normal distribution and plotted in the figures as mean±SEM. For each figure, n=the number of subjects. One-way ANOVA with Tukey's post hoc test was used for taxonomic comparisons from ITS sequencing analysis in FIG. 9B and co-culture assays in FIGS. 16 and 17. Data for plasma LBP in FIGS. 10A and 10B were analyzed by non-parametric Mann-Whitney test. Significant differences emerging from the above tests are indicated in the figures by *p<0.05, **p<0.01.

### Data and Software Availability

De-identified patient metadata, 16S, ITS and single-cell RNA sequencing data will be deposited to dbGAP and GenBank, and all code made available on Github.

### Example 6. Gnotobiotic (Germ-free) Mice Colonized with C. innocuum Exhibited Significant MAT Expansion

Experiment was carried out in 16-week old mice. Three mice were colonized with a standard “healthy” consortium of bacteria called Altered Schaedler's flora (ASF); two mice were colonized with ASF+C. innocuum; and another two mice were colonized with ASF+C. innocuum+dextran sodium sulfate (DSS). In the first group where ASF was colonized with no C. innocuum, the intestines were with no discernable MAT growth. In the second group where both ASF and C. innocuum were colonized, there is greater amounts of MAT surrounding the intestines. In the third group where the inflammatory chemical, DSS, was used to induce intestinal inflammation in mice colonized with both ASF and C. innocuum, there is a large amount of mesenteric adipose growth. The addition of DSS was used to mimic the inflamed intestines seen in Crohn's disease. But even without the DSS, the addition of C. innocuum resulted in greater MAT expansion.

When we performed this in much younger mice, we did not see such a pronounced adipose growth. This is likely the case in Crohn's disease too where in the pediatric resections, we see very little creeping fat.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method of conducting examination of a subject with an inflammatory bowel disease, comprising:
detecting for the presence or absence of a microorganism in a biological sample of the subject,
wherein the microorganism comprises one or more of Clostridium, Parabacteroides, Erysipelatoclostridium, Veillonella, and Bifidobacteria.

2. The method of claim 1, wherein the microorganism is selected from the group consisting of Clostridium innocuum, Parabacteroides distasonis, Erysipelatoclostridium ramosum, Clostridium symbiosum, Veillonella parvula, Bifidobacteria pseudolongum, and combinations thereof.

3. The method of claim 1, wherein the microorganism is Clostridium innocuum.

4. The method of claim 1, wherein the biological sample is stool, mucosal biopsy, mucosal wash, or adipose tissue of the subject, said adipose tissue is abdominal wall adipose tissue, creeping fat, or mesenteric adipose tissue.

5. The method of claim 1, wherein the subject is a human.

6. The method of claim 1, wherein the subject has Crohn's disease.

7. The method of claim 1, wherein the subject is susceptible to fibrosis or has undergone treatment or surgery of fibrosis removal.

8. A method of detecting fibrosis in a subject in need thereof, comprising:
detecting for the presence or absence of a microorganism in a biological sample of the subject, said microorganism comprises one or more of Clostridium, Parabacteroides, Erysipelatoclostridium, Veillonella, and Bifidobacteria,
wherein the presence of the microorganism indicates the presence or onset of the fibrosis.

9. The method of claim 8, wherein the microorganism is selected from the group consisting of Clostridium innocuum, Parabacteroides distasonis, Erysipelatoclostridium ramosum, Clostridium symbiosum, Veillonella parvula, Bifidobacteria pseudolongum, and combinations thereof.

10. The method of claim 8, wherein the microorganism is Clostridium innocuum.

11. The method of claim 8, wherein the biological sample is stool, mucosal biopsy, mucosal wash, or adipose tissue of the subject, said adipose tissue is abdominal wall adipose tissue, creeping fat, or mesenteric adipose tissue.

12. The method of claim 8, wherein the fibrosis is ileal fibrosis.

13. The method of claim 8, wherein the subject has Crohn's disease.

14. The method of claim 8, wherein the subject is suspected of developing fibrotic strictures.

15. A method of detecting a microorganism selected from the group consisting of Clostridium innocuum, Parabacteroides distasonis, Erysipelatoclostridium ramosum, Clostridium symbiosum, Veillonella parvula, and Bifidobacteria pseudolongum from a biological sample of a subject having an inflammatory bowel disease, comprising:
obtaining a biological sample from stool, mucosal biopsy, mucosal wash, or adipose tissue of the subject having an inflammatory bowel disease;
sequencing the biological sample for DNA, RNA, or both, of a microorganism selected from the group consisting of Clostridium innocuum, Parabacteroides distasonis, Erysipelatoclostridium ramosum, Clostridium symbiosum, Veillonella parvula, and Bifidobacteria pseudolongum.

16. The method of claim 15, further comprising cultivating a colony of microorganisms from the biological sample, wherein the sequencing step comprises sequencing the colony for the DNA, the RNA, or both.

17. The method of claim 15, further comprising measuring a level of expression of one or more genes or encoded proteins thereof comprising lysozyme, complement C1q A chain (C1QA), complement component 5a receptor 1 (C5AR1), toll-like receptor 2, collagen, lipopolysaccharide (LPS)-binding protein (LBP), or a combination thereof.

18. The method of claim 15, further comprising measuring a level of expression of one or more genes or encoded proteins thereof comprising an oxidative stress-related gene or protein, an adhesion-related gene or protein, an iron acquisition-related gene or protein, a lipid metabolism-related gene or protein, a metabolite, or a combination thereof.

19. The method of claim 18, wherein the oxidative stress-related gene or protein comprises superoxide dismutase, superoxide reductase, peroxiredoxin/thiol peroxidase, NADH oxidase, cystathionine beta-lyases/cystathionine gamma-synthases, manganese catalase, or Alkyl hydroperoxide reductase/anaerobic sulfite reductase; the adhesion-related gene or protein comprises ethanolamine permease, tryptophan synthase, peptidase M23/37, fibronectin, fibrinogen-binding protein, oligopeptide ABC transporter substrate-binding protein OppA, oligopeptide ABC transporter ATP-binding protein OppD, oligopeptide ABC transporter ATP-binding protein OppF, or type IV fimbriae; the iron acquisition-related gene or protein comprises ABC-type Fe3+ transport system or iron ABC transporter permease; the lipid metabolism-related gene or protein comprises monoglyceride lipase or lysophospholipase; and the metabolite comprises acyl-CoA: acetate CoA-transferase, acetate kinase, or tryptophan synthase.

20. The method of claim 15, wherein the subject has Crohn's disease.
